(19) United States
(12) Patent Application Publication
(10) Pub. No.: US 2026/0338728 A1
(43) Pub. Date: Oct. 29, 2026

(54) METHOD AND DEVICES FOR DETECTING A FIBRINOGEN LEVEL IN A SAMPLE

(71) Applicant: ROCHE DIAGNOSTICS OPERATIONS, INC., Indianapolis, IN (US)

(72) Inventors: Klaus Andreas BAUER-ESPINDOLA, Mannheim (DE); Sascha MEYER DOS SANTOS, Portimão (PT); Dagmar SCHUMACHER, Indianapolis, IN (US); Michael LAMBERTSON, Mannheim (DE)

(21) Appl. No.: 19/701,560

(22) Filed: Jun. 8, 2026

Related U.S. Application Data
(63) Continuation of application No. PCT/EP2024/084819, filed on Dec. 5, 2024.

(30) Foreign Application Priority Data
Dec. 6, 2023 (EP) 23214577.1

(51) Int. Cl. G01N 27/327; C12Q 1/00; C12Q 1/37; G01N 33/68

(52) U.S. Cl. CPC G01N 27/3275; C12Q 1/005; C12Q 1/37; G01N 33/6887; G01N 2333/75; G01N 2333/96433

(57) ABSTRACT

A test element for detecting a fibrinogen level in a sample of a bodily fluid is disclosed. The test element may include at least one substrate and at least one capillary for receiving and transporting the sample. The test element may further include at least one test region and at least one control region within the capillary, wherein the test element, in the test region, comprises at least two test electrodes, wherein the test element, in the control region, includes at least two control electrodes. The test element further includes, in the test region, at least one detector compound, the detector compound being capable of specifically cleaving fibrinogen.

### CROSS-REFERENCE TO RELATED APPLICATIONS

This application is a continuation of International Patent Application No. PCT/EP2024/084819, filed Dec. 5, 2024, which claims the benefit of priority to European Patent Application No. 23214577.1, filed Dec. 6, 2023, the disclosures of which are herein incorporated by reference in their entirety.

### TECHNICAL FIELD

The present disclosure relates to methods of ex-vivo detecting a fibrinogen level in a sample of a bodily fluid and to a method of manufacturing a test element for detecting a fibrinogen level in a sample of a bodily fluid (e.g., for use in medical diagnostics and treatment, particularly for point-of-care applications, such as in hospitals or other healthcare facilities.

### BACKGROUND

In the field of medical diagnostics, in many cases, one or more diagnostic parameters of a patient are measured. As an example, one or more diagnostic parameters have to be detected in samples of bodily fluid, such as blood, interstitial fluid, urine, salvia, or other types of bodily fluid. Examples of diagnostic parameters to be detected are glucose, triglycerides, lactate, cholesterol, coagulation parameters, such as fibrinogen, or other types of diagnostic parameters typically present in these bodily fluids. An appropriate treatment may be chosen based on the concentration, the presence and/or the temporal development of the diagnostic parameter, if necessary. Patients undergoing large surgical interventions (e.g., trauma patients) with major bleeding risks, like cardiac surgery or larger abdominal surgeries, or women post partem, are at risk of bleeding complications mainly due to a strong decrease in fibrinogen levels. Determining clottable fibrinogen can help to peri- or postoperatively identify patients at risk and may also control therapeutic interventions with, e.g., fibrinogen concentrate or fresh frozen plasma. Besides the situations mentioned, other situations and medical indications may exist in which the level of clottable fibrinogen in bodily fluids is a valuable biomedical indicator.

Batroxobin as the active compound of the snake venom of Bothrops atrox has been known for many decades. Further, methods and devices making use of batroxobin have also been described. Thus, Heimann et al. (J Clin Chem Cin Biochem, 1979 June; 17(6):369-72) disclose that fibrinogen can be precipitated from Heparin-plasma by Reptilase (Batroxobin). The electrophoretic pattern of the supernatant on cellulose acetate is nearly identical with that of the corresponding serum. This technique is also useful in differentiating a gradient of fibrinogen (caused by effects of anticoagulants) from a myeloma peak. Further, Vu et al. (The Journal of Biological Chemistry Vol. 288, No. 23, pp. 16862-16871, Jun. 7, 2013) describe in their study that Batroxobin is a thrombin-like serine protease from the venom of Bothrops atrox moojeni that clots fibrinogen. In contrast to thrombin, which releases fibrinopeptide A and B from the NH₂-terminal domains of the Aα- and Bβ-chains of fibrinogen, respectively, batroxobin only releases fibrinopeptide A. Because the mechanism responsible for these differences is unknown, Vu et al. compared the interactions of batroxobin and thrombin with the predominant γA/γA isoform of fibrin(ogen) and the γA/γ′ variant with an extended γ-chain. Moreover, You et al. (FEBS Letters 571 (2004) 67-73) disclose that a thrombin-like enzyme of Bothrops atrox moojeni venom, batroxobin, specifically cleaves fibrinogen a chain, resulting in the formation of non-crosslinked fibrin clots. They report that the cDNA encoding batroxobin was cloned, expressed in Pichia pastoris and the molecular function of purified recombinant protein was also characterized. The recombinant batroxobin had an apparent molecular weight of 33 kDa by SDS-PAGE analysis and biochemical activities similar to those of native batroxobin. The purified recombinant protein strongly converted fibrinogen into fibrin clot in vitro, and shortened bleeding time and whole blood coagulation time in vivo. However, it did not result in any considerable alterations of other blood coagulation factors. Several lines of experimental evidence in this study suggest that the recombinant batroxobin is a potent procoagulant agent.

Stürzebecher et al. (Toxicon. Vol, 24, No. 6, pp. 585-595, 1986) disclose that benzamidine derivatives which are competitive inhibitors of trypsin-like serine proteinases also inhibited the enzymatic activity of batroxobin, a thrombin-like snake venom proteinase. Structure-activity relationships showed that primary amides of 4-amidinophenyl-α-aminobutyric acid have pronounced, relatively selective antibatroboxin activity. Identical effects were found on batroxobin isolated from the venoms of Bothrops atrox or Bothrops moojeni. Esters containing a benzamidine moiety acylated the active centre serine hydroxyl of either batroxobin, however, the inhibition was temporary. Such compounds, especially 4-amidinophenyl esters of substituted benzoic acids, are a particularly useful tool for designing acyl-batroxobin intermediates with different deacylation rates. With 4-nitrophenyl 4′-guanidinobenzoate, the acyl enzyme was formed so rapidly that titration of the active site of batroxobin was possible. Irreversible inhibition of batroxobin was caused only by the selective thrombin inhibitor D-Phe-Pro-ArgCH₂CI.

Further, Harris et al. (Trends in Analytical Chemistry 50 (2013) 85-95) report that automated technologies have revolutionized the monitoring of coagulation disorders in the central hospital laboratory setting, allowing for high throughput testing, improved accuracy and precision, accompanied by a marked reduction in human error. However, they still require trained operators and sample transportation. With the advent of point of care (POC) testing, the working principle of traditional coagulometers was used as the foundation for the development of miniaturised devices. A number of POC coagulation devices have been commercially available for many years now, allowing the patient to assume more control over the management of their own medication, e.g. warfarin. While POC devices for measuring anticoagulation have relied principally on clotting time tests, novel platelet function tests, and factor-specific assays based on enzymatic or immunoassay principles are becoming available, driven by the emergence of new anticoagulant drugs, in addition to the inability of clotting tests to accurately detect many thrombotic disorders. In their review, Harris et al. highlight recent progress in the development of POC coagulation monitoring technologies and examines their future potential in clinical diagnostics.

WO 2019/068940 A1 describes a method for measuring the fibrinogen level in a sample, which is particularly useful in emergency situations. The method is independent of thrombin formation and is not interfered by the presence of oral anti-coagulation drugs or other chemicals contrary to the commonly used clotting assays. US 2021/0016275 A1 describes assay devices, methods and microfluidic cartridges for analysis of fluids, such as whole blood. A fibrinogen assay cartridge is adapted to measure whole blood flow rates on exposure to thrombin and measures hematocrit for a plasma fibrinogen calculation. Multiple channel cartridges are provided to allow determination of multiple assays (e.g., coagulation panel) from a single sample on a single cartridge.

### BRIEF SUMMARY

The following passages introduce a selection of concepts in a simplified form that are further described below in the Detailed Description. This summary of selected aspects is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

Despite the advantages achieved by current coagulation monitoring technologies, several technical challenges remain. In particular, low sensitivity, specifically in the regime of low fibrinogen levels, remains a challenge for current devices and methods. This problem is particularly relevant for assays relying on an inversely proportional relationship between the fibrinogen level and the detectable signal. Further disadvantages of known methods and devices based on batroxobin often include a considerable turnaround time and low user-friendliness. Such methods and devices also often require stationary, non-portable systems. Furthermore, known methods and devices measuring clot formation are usually not fibrinogen-specific but depend on further coagulation factors, that is coagulation factors other than fibrinogen

As such, there remains a need in the art for methods and devices that address the above-mentioned technical challenges. To that end, the present disclosure provides, among other inventive aspects, methods and devices which allow for a detection of fibrinogen levels in samples of bodily fluids at a high sensitivity in a fast and user-friendly fashion. In some aspects, such methods and/or devices include a test element for detecting a fibrinogen level in a sample, as well as test systems, methods of ex-vivo detecting a fibrinogen level in a sample, and methods of manufacturing a test element. Advantageous embodiments which may be realized in an isolated fashion or in any arbitrary combinations are provided herein and illustrated by the accompanying drawings.

### BRIEF DESCRIPTION OF THE DRAWINGS

Various features and advantages of aspects of the present disclosure will be more fully understood from the following detailed description of illustrative embodiments taken in conjunction with the accompanying drawings in which:

FIGS. 1A-1B show a schematic top view of an example of a test element according to the present disclosure.

FIGS. 2A-2B show a schematic cross-sectional view of an embodiment of an impedance test system (FIG. 2A) and of an embodiment of the test element (FIG. 2B).

FIGS. 3A-3B show a measurement diagram of an electrical impedance control measurement (FIG. 3A) and an electrical impedance test measurement (FIG. 3B).

FIGS. 4A-4B show a measurement diagram of an electrical impedance test measurement acquired at an alternating voltage of 10 kHz (FIG. 4A) and 100 kHz (FIG. 4B).

FIGS. 5A-15 show a measurement diagram of an electrical impedance test measurement acquired at an alternating voltage of 10 kHz (FIG. 4A) and 100 kHz (FIG. 4B), both with a plurality of replicates.

FIGS. 6A-6B each show a measurement diagram wherein a normalized impedance test measurement is plotted against a base 10 logarithm of the frequency of the alternating voltage in an overview format (FIG. 6A) and in a detailed view (FIG. 6B).

FIGS. 7A-7B show a measurement diagram of an electrical impedance control measurement (FIG. 7A) and an electrical impedance test measurement (FIG. 7B).

FIGS. 8A-8B show a measurement diagram with the three shifted impedance test measurements (FIG. 8A) and a fit curve based thereon (FIG. 8B).

FIG. 9 shows a measurement diagram with a plurality of shifted impedance test measurements.

FIG. 10 shows a measurement diagram of an electrical impedance test measurement in the presence and in the absence of a fibrin polymerization blocker.

FIG. 11 shows a flowchart of an impedance method of ex-vivo detecting a fibrinogen level in a sample of a bodily fluid.

FIG. 12 shows a flowchart of a method of manufacturing a test element for detecting a fibrinogen level in a sample of a bodily fluid.

FIG. 13 shows an embodiment of a resistance test system for detecting a fibrinogen level in a sample of a bodily fluid.

FIG. 14 shows a flow chart of an embodiment of a resistance method of ex-vivo detecting a fibrinogen level in a sample of a bodily fluid.

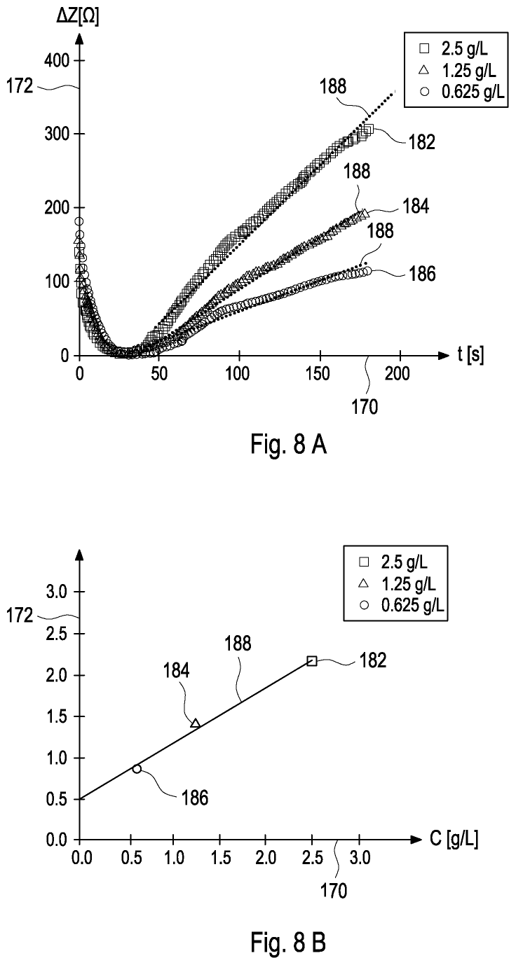

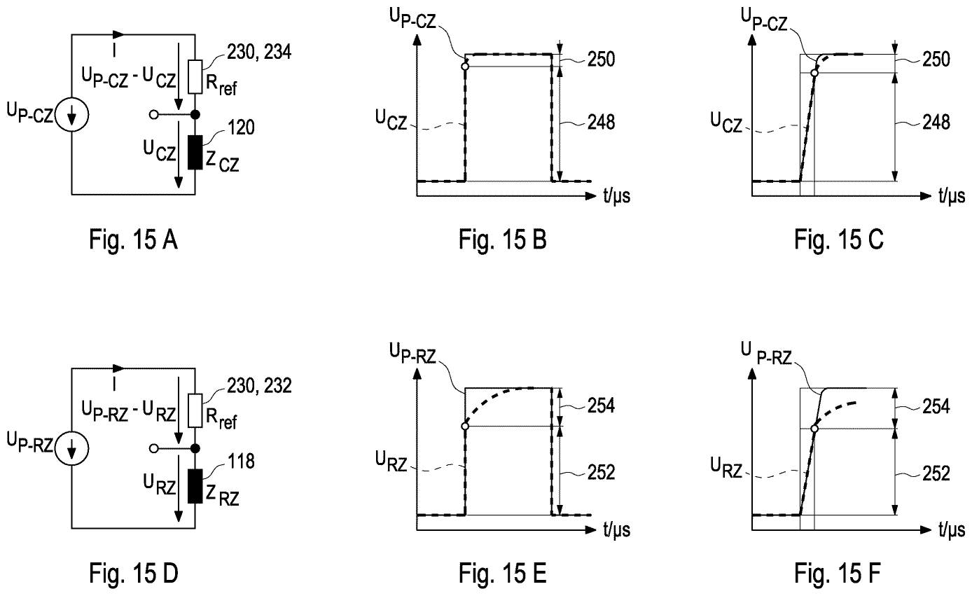

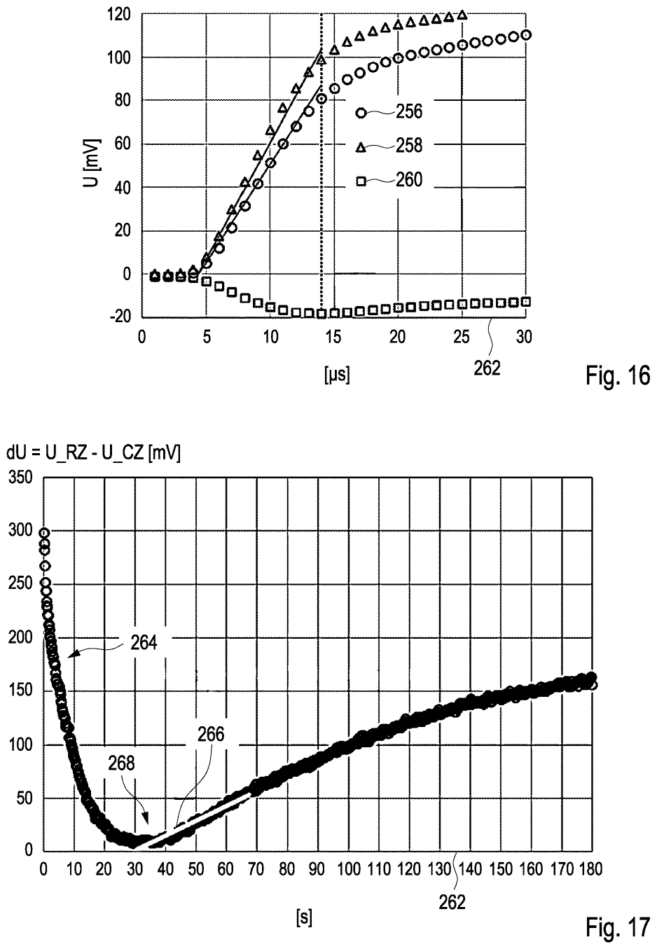

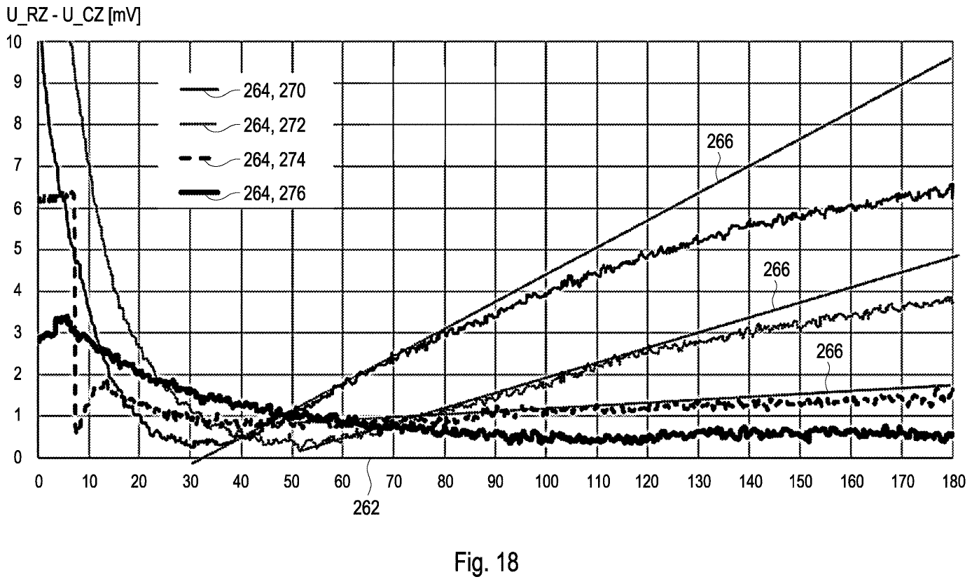

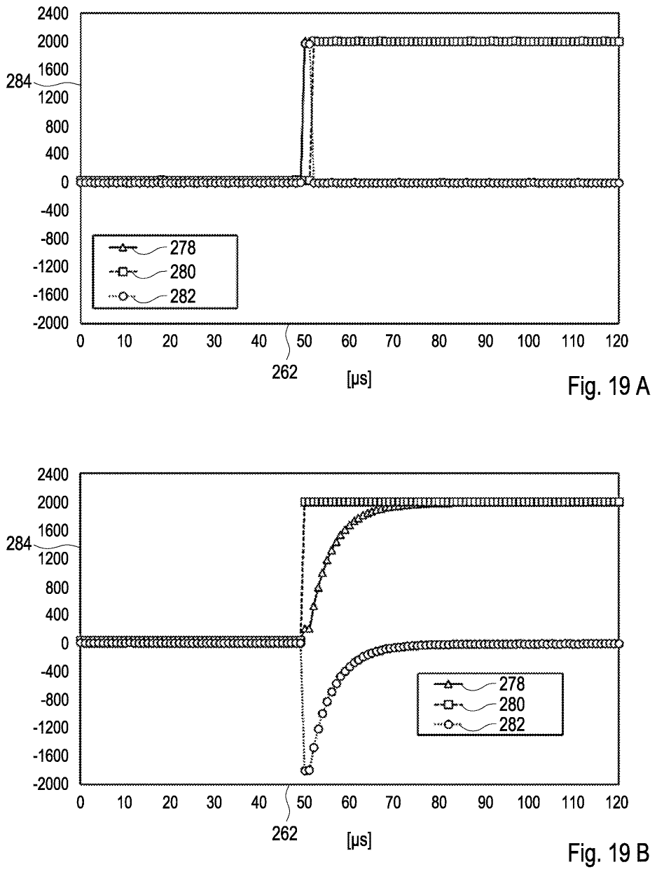

FIGS. 8A-8B show equivalent circuits of the control region (FIG. 15A) and the test region (FIG. 15D) and respective diagrams of voltage signals (FIG. 15B and FIG. 15E) and response signals (FIG. 15C and FIG. 15F).

FIG. 16 shows a diagram of test response signals and control response signals;

FIG. 17 shows a diagram of a plurality of averaged minima of a difference between a plurality of test response signals and a plurality of control response signals;

FIG. 18 shows a diagram of a plurality of averaged minima for different fibrinogen levels; and

FIGS. 19A-19B show diagrams of determining a wetting of control electrodes and test electrodes.

### DETAILED DESCRIPTION

The present disclosure relates to methods, assays, devices, and kits for measuring fibrinogen in a sample (e.g., blood or another biological fluid or fraction thereof). The inventive embodiments described herein enable accurate and easily deployable point-of-care devices to assay fibrinogen in a clinical setting, promoting improved patient care and therapeutic outcomes.

Prior to describing the various embodiments, the following definitions are provided and should be used unless otherwise indicated.

It is to be understood that as used in the specification and in the claims, “a” or “an” can mean one or more, depending upon the context in which it is used. Thus, for example, reference to “an” item cell” can mean that at least one item can be utilized.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The various devices, systems and methods described herein may, as the skilled person will recognize, be performed by using alternative features. Similarly, features introduced by “in an embodiment of the invention,” “in an embodiment of the disclosure,” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of the present disclosure, a test element for detecting a fibrinogen level in a sample of a bodily fluid is described. The test element comprises at least one substrate and at least one capillary for receiving and transporting the sample. The test element further comprises at least one test region and at least one control region within the capillary. The test element comprises, in the test region, at least two test electrodes. The test element further comprises, in the control region, at least two control electrodes. In the test region, the test element further comprises at least one detector compound, the detector compound being capable of specifically cleaving fibrinogen.

The term “test element” as used herein is a broad term and is to be given its ordinary and customary meaning to a person of ordinary skill in the art and is not to be limited to a special or customized meaning. The term specifically may refer, without limitation, to an arbitrary device which is capable of measuring or detecting at least one analyte in a sample and/or at least one parameter of the sample or a component of the sample, such as in a sample of bodily fluid. The test element may specifically be suited for an ex-vivo measurement, specifically an in-vitro measurement. The test element may specifically be a strip-shaped test element. As used herein, the term “strip-shaped” refers to an element having an elongated shape and a thickness, wherein an extension of the element in a lateral dimension exceeds the thickness of the element, such as by at least a factor of 2, preferably by at least a factor of 5, more preferably by at least a factor of 10 and most preferably by at least a factor of 20 or even at least a factor of 30. Thus, the test element specifically may also be embodied as or referred to as a “test strip”.

The term “fibrinogen level” as used herein is a broad term and is to be given its ordinary and customary meaning to a person of ordinary skill in the art and is not to be limited to a special or customized meaning. The term specifically may refer, without limitation, to a measurand or a measurement value indicating a fibrinogen-related or fibrinogen-based quality of the sample, such as a concentration of fibrinogen in the sample, an amount of fibrinogen in the sample, a presence or an absence of fibrinogen in the sample. Thus the term “detecting a fibrinogen level” may refer, without limitation, to a quantitative and/or qualitative determination of fibrinogen in the sample. In particular, a measured value indicating a fibrinogen level may be determined as part of the detection. The result of the detection may for example be a fibrinogen concentration. The fibrinogen level in the sample may be determined in a direct and/or in an indirect manner. In particular, the level of fibrinogen may be deduced or inferred from at least one fibrinogen-based or fibrinogen-related quality or parameter of the sample, which may be measured as part of the detection process. The detection of fibrinogen may particularly be based on the specific quality of fibrinogen to produce clottable fibrin upon appropriate cleavage.

The term “bodily fluid” as used herein is a broad term and is to be given its ordinary and customary meaning to a person of ordinary skill in the art and is not to be limited to a special or customized meaning. The term specifically may refer, without limitation, to a liquid, which is typically present in a body or a body tissue of a user or patient and/or may be produced by the body of the user or the patient. Preferably, the bodily fluid may be selected from the group consisting of blood and interstitial fluid. However, additionally or alternatively, one or more other types of bodily fluids may be used, such as at least one of saliva, lacrimal fluid, urine or other bodily fluids.

The term “sample of the bodily fluid” as used herein is a broad term and is to be given its ordinary and customary meaning to a person of ordinary skill in the art and is not to be limited to a special or customized meaning. The term specifically may refer, without limitation, to an arbitrary aliquot part or aliquant part of a biological fluid which directly is a bodily fluid as defined above or which is derived from a bodily fluid, such as by one or more pre-processing steps, e.g. by centrifugation. Specifically, the sample of the bodily fluid may refer to a part of the bodily fluid that is applied to the test element, more specifically, that is received by the capillary. Thus, as an example, the sample of the bodily may be a droplet of the bodily fluid as gathered from the body of a person, such as a droplet of blood and/or interstitial fluid generated, e.g., by piercing of a skin portion of the person, e.g. with a lancet, a needle or the like. The sample of bodily fluid may also be simply referred to as the sample.

The term “substrate” as used herein is a broad term and is to be given its ordinary and customary meaning to a person of ordinary skill in the art and is not to be limited to a special or customized meaning. The term specifically may refer, without limitation, to an arbitrary element designed to carry one or more other elements disposed thereon or therein. Particularly, the substrate may be a planar substrate. The substrate may, specifically, have an elongated shape, such as a strip shape or a bar shape; however, other kinds of shapes may also be feasible. The substrate may comprise at least partially or even completely, at least one electrically insulating material, especially in order to avoid unwanted currents between electrically conducting elements as carried by the substrate. By way of example, the electrically insulating material may be selected from polyethylene terephthalate (PET) or polycarbonate (PC); however, other kinds of electrically insulating materials may also be feasible. The substrate may in particular be flexible.

The term “capillary” as used herein is a broad term and is to be given its ordinary and customary meaning to a person of ordinary skill in the art and is not to be limited to a special or customized meaning. The term specifically may refer, without limitation, to an arbitrary device configured for conducting a liquid sample along a predetermined path. In particular, upon entry into the capillary, the liquid sample may flow through the capillary, thus being transported by the capillary along the predetermined path. The transport of the sample through the capillary may in particular be at least partially promoted by capillary force. The capillary may specifically be a tube, in particular a small tube, such as a tube with a limited height and/or with a limited cross section or diameter, as will be described in more detail further below. The capillary, in particularly the tube, may be at least partially formed by the substrate and/or other layers of the test element as will also be described further below.

The term “test region” as used herein is a broad term and is to be given its ordinary and customary meaning to a person of ordinary skill in the art and is not to be limited to a special or customized meaning. The term specifically may refer, without limitation, to an arbitrary section of the test element, specifically to a section of the capillary, wherein the test region is configured for performing at least one measurement on the sample in the presence of the detector compound. Thus, the test region specifically may be a section or region of the test element, specifically of the capillary of the test element, comprising the at least one detector compound. A spatial extension of the test region, thus, may be defined by a spatial extension of the detector compound and/or by a spatial extension of one or more other components taking part in the detecting of the fibrinogen level in the sample of a bodily fluid, such as one or more of the test electrodes, specifically covered by the at least one detector compound. The measurement carried out in the test region may be part of a detection process for detecting the fibrinogen level in the sample. Thus, measurement values determined by the measurement in the test region may subsequently be processed further for detecting the fibrinogen level.

The test element comprises the at least two test electrodes and the at least two control electrodes. The term “electrode” as used herein is a broad term and is to be given its ordinary and customary meaning to a person of ordinary skill in the art and is not to be limited to a special or customized meaning. The term specifically may refer, without limitation, to an arbitrary electrically conductive element to which an electrical voltage or an electrical current is applicable. As an example, the electrode may comprise at least one layer, specifically at least one patterned layer, of at least one electrically conductive material on at least one substrate, such as at least one metal layer and/or at least one layer of an electrically conductive plastic material and/or electrically conductive polymer. The metal layer, as an example, may comprise at least one of: gold, silver copper, nickel, lead, tin, zinc, palladium, platinum, chromium, aluminum, magnesium, steel and alloys of all named metals. The layer may also comprise at least one non-metallic conductive material, e.g. graphite, graphene, carbon black, carbon nanotubes, boron doped diamond and compositions of graphene, graphite, carbon black, carbon nanotubes, conductive polymers, boron doped diamond (=BDD), nitrogen doped diamond (=NDD) and phosphorous doped diamond (=PDD). The electrode may specifically be configured to perform or take part in at least one electronic measurement of an element in direct or indirect electrical contact with the electrode, in particular the sample. The electrode may particularly be configured to apply a current and/or a voltage, more specifically an electrical potential, to the element in electrical contact with the electrode, in particular the sample. It shall be noted that when referring to an electrical voltage or an electrical current, the voltage may be a direct voltage or an alternating voltage and the current may be a direct current or an alternating current.

The term “test electrode” as used herein is a broad term and is to be given its ordinary and customary meaning to a person of ordinary skill in the art and is not to be limited to a special or customized meaning. The term specifically may refer, without limitation, to an arbitrary electrode located in the test region of the test element. The test region may be configured for performing the measurement on the sample in the presence of the detector compound. The measurement may specifically be performed using the test electrode. In particular, the detector compound may be directly or indirectly disposed on the test electrodes, e.g. by directly or indirectly depositing at least one layer of the detector compound onto the test electrodes. Additionally or alternatively, as will be outlined in further detail below, the detector compound may be disposed e.g. on at least one side wall and/or on at least one top or bottom wall of the capillary in the test region in which also the test electrodes are located, such that the detector compound reacts with the sample in the test region of the capillary and the test electrodes in the test region may detect the reaction.

The term “control region” as used herein is a broad term and is to be given its ordinary and customary meaning to a person of ordinary skill in the art and is not to be limited to a special or customized meaning. The term specifically may refer, without limitation, to an arbitrary section of the test element, wherein the control region is configured for performing at least one measurement on the sample in the absence of the detector compound, also referred to as a control measurement. Particularly, the test element, in the control region, may be devoid of the at least one detector compound. The measurement carried out in the control region may also be part of the detection process for detecting the fibrinogen level in the sample. Thus, measurement values determined by the measurement in the control region may then be further processed, e.g. together with the measurement values determined by the measurement in the test region, for detecting the fibrinogen level.

The term “control electrode” as used herein is a broad term and is to be given its ordinary and customary meaning to a person of ordinary skill in the art and is not to be limited to a special or customized meaning. The term specifically may refer, without limitation, to an arbitrary electrode located in the control region of the test element. The control region may be configured for performing the measurement on the sample in the absence of the detector compound. The measurement in the control region may specifically be performed using the control electrode. The control electrode specifically may be an uncoated electrode, specifically devoid of the detector compound. Further, the control electrodes may be located in the control region which may be entirely devoid of detector compound.

The term “detector compound” as used herein is a broad term and is to be given its ordinary and customary meaning to a person of ordinary skill in the art and is not to be limited to a special or customized meaning. The term specifically may refer, without limitation, to an arbitrary chemical or biological substance, e.g. a single substance or a mixture of substances, such as a molecule or a macromolecule, capable of specifically cleaving fibrinogen. The detector compound may specifically be a macromolecule with enzymatic activity, e.g. an enzyme, specifically a protein. The at least one detector compound may be used by itself or in conjunction with one or more further materials. Thus, as an example, the detector compound may be comprised in at least one matrix material, such as an inert matrix material.

The term “specifically cleaving fibrinogen” as used herein is a broad term and is to be given its ordinary and customary meaning to a person of ordinary skill in the art and is not to be limited to a special or customized meaning. The term specifically cleaving may refer, without limitation, to the capability of the detector compound to specifically bind to and cleave fibrinogen in a predetermined manner. Hence, said detector compound shall specifically bind to fibrinogen, i.e. shall not bind to other compounds. Moreover, the detector compound shall cleave fibrinogen in a specific manner, i.e. at at least one predetermined cleaving site. In particular, the detector compound may be capable of cleaving fibrinogen in such a way as to produce biologically active fibrin from its inactive precursor fibrinogen by releasing fibrinopeptide A. The process of cleaving fibrinogen may therefore essentially be an activation process. The result of the activation process may be the presence of biologically active fibrin, whose polymerization may trigger or take part in clot-formation as part of the coagulation process. In particular, the detection of the fibrinogen level in the sample of bodily fluid may be based on or related to the fibrin-induced clot-formation.

The activation process may be the basis and/or the trigger of the subsequent detection process, in which fibrinogen levels are detected in the sample of bodily fluid. In particular, the capability of specifically cleaving fibrinogen may distinguish the detector compound from other molecules or enzymes with cleaving properties, which may be capable of cleaving fibrinogen in an unspecific manner, e.g. at random cleaving sites or at cleaving sites that do not lead to the production of fibrin. Thus, in the context of the present disclosure the term “specifically cleaving fibrinogen” is to be understood as a specific way or a specific fashion of cleaving fibrinogen, the specific way being described in detail above and in further detail below. The term “specifically cleaving” fibrinogen, in the context of the present disclosure, is not to be understood as describing an optional feature or an optional quality of the detector compound but as specifying the detector compound by describing its specific quality.

The detector compound may in particular be a serine endopeptidase (E.C.3.4.21). Thus, the detector compound may specifically break peptide bonds of nonterminal amino acids. Said serine endopeptidase may specifically be a thrombin-like serine protease. In particular, the detector compound may specifically break down soluble fibrinogen into insoluble strands of fibrin. Said thrombin-like serine protease may have venombin A activity. Specifically, the detector compound may selectively cleave an Arginine bond in fibrinogen, to form fibrin, and release fibrinopeptide A. Particularly, said thrombin-like serine protease may be a batroxobin.

The test element comprises, in the test region, the at least one detector compound. The detector compound may be disposed on the test electrodes. Additionally or alternatively, the detector compound may be disposed on at least one side wall of the capillary, specifically on at least one side wall extending over the entire length of the test region in a direction of longitudinal extension of the capillary. Specifically, the detector compound may be comprised in at least one detector compound layer. Thus, the detector compound may be applied to the test electrodes and/or to the capillary in the form of a film or a layer that may at least partially cover or coat the test electrodes and/or the capillary, specifically in the test region. The detector compound layer and, optionally at least one control layer in the control region, may comprise at least one further substance apart from the detector compound, e.g. at least one matrix substance, at least one substance that may facilitate the application of the detector compound and/or the detector compound layer, at least one substance that may facilitate the adherence of the detector compound layer and the control layer, at least one substance that may support a stability of the detector compound in the detector compound layer, or any combination of at least two of these possibilities and/or one or more further substances. The detector compound layer and the control layer may also comprise substances which stabilize pH, ion strength and/or provide a constant base conductivity. The detector compound layer and the control layer may also comprise colored substances which support a coating process.

In case the detector compound is comprised in at least one detector compound layer comprising, besides the at least one detector compound, at least one further substance, e.g. a matrix substance or the like, the at least one further substance may also be applied to the control region, in order to provide comparable properties of the test region and the control region. Thus, the test region may comprise the at least one detector compound layer having the at least one detector compound and the at least one further substance, and the control region may comprise at least one control layer, wherein the control layer may comprise the at least one further substance without the detector compound. The detector compound layer and the control layer, as an example, may have identical or at least essentially identical properties, e.g. with respect to their respective layer thicknesses, surface polarities or the like, except for the fact that the detector compound layer comprises the detector compound whereas the control layer is devoid of the detector compound. The term “essentially”, as used herein, specifically may comprise tolerances of no more than 10%, specifically of no more than 5%. Specifically, the detector compound layer and the control layer may comprise, besides the detector compound in the detector compound layer, the same substances, e.g. the same matrix material, in order to provide identical or at least essentially identical properties.

Thus, the at least one control layer may be applied to the control electrodes and/or to at least one section of the control region, e.g. to at least one side wall of the capillary in the control region, wherein the control layer may be devoid of the detector compound. Thus, the control layer in the control region may comprise all substances or components present in the detector compound layer in the test region except for the detector compound. Thus, the control layer may infer comparable properties to both the test region, specifically the test electrodes, and the control region, specifically the control electrodes, except for the capability of specifically cleaving fibrinogen, which is reserved to the test region, specifically the test electrodes, due to the presence of the detector compound. Other properties, however, e.g. flow characteristics and other properties may be comparable or even identical.

The test element comprises the test region and the control region. The test region and the control region may specifically be disposed in the test element in accordance with a setup selected from the group consisting of:
the test region and the control region are disposed in the same capillary, wherein the control electrodes and the test electrodes are disposed within the capillary such that the sample of bodily fluid entering the capillary passes the control electrodes before passing the test electrodes; and
the test element comprises at least two capillaries, wherein the test region and the control region are disposed in different capillaries, specifically in different capillaries having a common application opening.

Thus, in the first setup as described above, the sample of bodily fluid may first pass the control region before it then passes the test region, such that the sample passes the two regions in a subsequent manner. Alternatively, as described in the second setup above, the sample of bodily fluid may be received by the common application opening, then flow into the two capillaries. In this setup, the sample may be subdivided into two portions, wherein one passes the control region while the other passes the test region. In this setup the common application opening may be described or referred to as a common capillary, a common section of the capillary or a common capillary active opening for sample application and the optional at least two different capillaries may be described or referred to as two subcapillaries branching out from the common capillary.

The test region and the control region may be disposed in the same capillary as described above. In this setup, the control electrodes and the test electrodes may specifically be spaced apart by a minimum distance of 1.0 mm to 2.5 mm, specifically by a minimum distance of 1.6 mm to 2.0 mm. Within typical reaction times, this separation may specifically be suited for avoiding a cross-talk from the detection reaction in the test region into the control region, such that the control measurement in the control region may remain unaffected by the reaction in the test region. Alternatively, the test element may comprise at least two capillaries, with the test region and the control region being disposed in different capillaries as also described above. In this setup, a minimum distance between at least one application opening of the capillaries and the control electrodes and a minimum distance between the at least one application opening and the test electrodes may be essentially identical. The term “essentially identical”, as used herein and as used below, specifically may comprise tolerances of no more than 10%, specifically of no more than 5%. Thus, when applying the sample to the application opening the sample may flow through the two different capillaries and reach the test region and the control region disposed in the different capillaries simultaneously. Specifically, the test element may comprise at least two capillaries, with the test region and the control region being disposed in different capillaries, wherein the capillaries may comprise a common application opening.

In particular, the test element may be a strip-shaped test element, wherein the substrate may be a flexible substrate. The test element may further comprise at least one cover layer and at least one spacer layer disposed between the substrate and the cover layer, wherein the spacer layer may form sidewalls of the capillary. The test element may further contain at least one application opening of the capillary and may further contain at least one vent opening of the capillary, wherein the test region and the control region may be disposed in between the application opening and the vent opening. The test element further comprises the test electrodes and the control electrodes. The test electrodes and the control electrodes may be disposed on the substrate, specifically on the same side of the same substrate. Further, the test region and the control region may have essentially identical dimensions. Furthermore, the test electrodes and the control electrodes may have an essentially identical geometry. The test electrodes may in particular contain test electrode fingers spaced apart in a direction of longitudinal extension of the capillary. The test electrode fingers may specifically be spaced apart by 0.10 mm to 1.00 mm, specifically by 0.20 mm to 0.80 mm. The test electrode fingers may particularly have a width of 0.1 mm to 0.5 mm, specifically of 0.25 mm to 0.4 mm. The test electrodes may specifically comprise two test electrodes, the two test electrodes having interleaving test electrode fingers. Further, the control electrodes may contain control electrode fingers spaced apart in a direction of longitudinal extension of the capillary. The control electrode fingers may specifically be spaced apart by 0.10 mm to 1.00 mm, specifically by 0.20 mm to 0.80 mm. In particular, the control electrode fingers may have a width of 0.1 mm to 0.5 mm, specifically of 0.25 mm to 0.4 mm. The control electrodes may specifically comprise two control electrodes, the two control electrodes having interleaving control electrode fingers. As will be shown in further detail below, these dimensions and/or geometries specifically are suited for performing impedance measurements in samples of blood and/or interstitial fluid, generating a sufficient signals and sufficient signal-to-noise ratio.

The test electrodes and the control electrodes may comprise contact leads having contact pads. The contact pads of the test electrodes and the control electrodes may particularly be disposed on the same side of the substrate. Specifically, at least a first one of the test electrodes and at least a first one of the control electrodes may be electrically contacted via a common contact lead, wherein at least a second one of the test electrodes and at least a second one of the control electrodes may be electrically contacted via separate contact leads. Thereby, the number of electrical contact leads may be reduced, which specifically may save space and costs.

The test element further comprises the capillary. The capillary may in particular have a width of 0.5 mm to 2.5 mm, specifically of 1.0 to 2.0 mm, more specifically of 1.5 mm. Specifically, the capillary may have a height of 50 μm to 500 μm, specifically of 100 μm to 300 μm, more specifically of 175 μm. These dimensions are suited, specifically, for conducting bodily fluids such as blood and/or interstitial fluid by capillary forces.

In a further aspect of the present disclosure, an impedance test system for detecting a fibrinogen level in a sample of a bodily fluid is described. The impedance test system comprises:
a) at least one test element as described herein, such as in any of the embodiments described above and/or as in any one of the embodiments described in further detail below; and
b) at least one test device having contacts for electrically contacting the test element, the test device further having at least one impedance measurement unit, wherein the impedance measurement unit is configured for performing at least one electrical impedance test measurement using the test electrodes, wherein the impedance measurement unit is further configured for performing at least one electrical impedance control measurement using the control electrodes.

The test element comprised by the impedance test system is a test element according to the present disclosure. Thus, regarding the test element, its definitions and embodiments, reference is made to the test description of the test element above and as described in further detail below. With respect to further terms, definitions and embodiments referred to in the context of the impedance test system, reference is also made to the description of the test element above and as described in further detail below.

The term “system” as used herein is a broad term and is to be given its ordinary and customary meaning to a person of ordinary skill in the art and is not to be limited to a special or customized meaning. The term specifically may refer, without limitation, to an arbitrary set of interacting or interdependent components parts forming a whole. Specifically, the components may interact with each other in order to fulfill at least one common function. The at least two components may be handled independently or may be coupled or connectable. The term “test system” as used herein is a broad term and is to be given its ordinary and customary meaning to a person of ordinary skill in the art and is not to be limited to a special or customized meaning. The term specifically may refer, without limitation, to an arbitrary system configured for performing at least one test and/or at least one analysis, e.g. an analysis of a sample. As part of the analysis at least one analyte in the sample and/or at least one parameter of the sample, e.g. a diagnostic parameter, may be determined. The components of the test system may in particular comprise the test element and the test device. The test system may comprise further components. The term “impedance test system” as used herein is a broad term and is to be given its ordinary and customary meaning to a person of ordinary skill in the art and is not to be limited to a special or customized meaning. The term specifically may refer, without limitation, to an arbitrary test system configured for performing at least one impedance test and/or at least one impedance analysis, e.g. an impedance analysis of a sample.

The term “test device” as used herein is a broad term and is to be given its ordinary and customary meaning to a person of ordinary skill in the art and is not to be limited to a special or customized meaning. The term specifically may refer, without limitation, to an arbitrary functional element configured for interacting with the test element in such a way as to determine the fibrinogen level of the sample of bodily fluid applied to the test element. The test device comprises contacts for electrically contacting the test element.

Thus, the test device and the test element may be electrically connectable, in particular via the contacts of the test device. Specifically, an electrical connection between the test device and the electrodes of test element may be establishable via the contact pads of the contact leads being contacted by the contacts of the test device. Further, an electrical current and/or an electrical voltage, more specifically an electrical potential, may be applied to the electrodes of the test element using the test device.

The term “contact” as used herein, also referred to as an “electrical contact”, is a broad term and is to be given its ordinary and customary meaning to a person of ordinary skill in the art and is not to be limited to a special or customized meaning. The term specifically may refer, without limitation, to an arbitrary functional element of electrically conductive material configured for establishing an electrical connection between a first object comprising the contact and at least one further object. The electrical connection may be establishable by bringing the first object into close proximity with the second object, such that the second object physically touches the contact.

The term “measurement unit” as used herein is a broad term and is to be given its ordinary and customary meaning to a person of ordinary skill in the art and is not to be limited to a special or customized meaning. The term specifically may refer, without limitation, to an arbitrary functional element configured for performing or controlling at least one measurement. Accordingly, the term “impedance measurement unit” as used herein is a broad term and is to be given its ordinary and customary meaning to a person of ordinary skill in the art and is not to be limited to a special or customized meaning. The term specifically may refer, without limitation, to an arbitrary measurement unit configured for performing or controlling at least one impedance measurement. The impedance measurement unit may be configured for controlling further functional elements that may take part in the impedance measurement. The test device, in particular the impedance measurement unit, may in particular comprise at least one potentiostat and/or at least one bipotentiostat and/or at least one polypotentiostat. Additionally or alternatively, the test device, in particular the impedance measurement unit, may, in particular comprise at least one current source and/or at least one voltage source, for applying at least one of a current and a voltage to the test electrodes and/or the control electrodes, respectively, more specifically at least one alternating current and/or at least one alternating voltage. Further, the test device, in particular the impedance measurement unit, may, in particular, comprise at least one of a voltage measurement device and/or a current measurement device, specifically for measuring at least one of a current and a voltage of the test electrodes and/or the control electrodes, respectively. Specifically, the test device, in particular the impedance measurement unit, may be configured for measuring at least one system response of the control electrodes and/or the test electrodes, the system response being at least one of a current signal and a voltage signal measured at at least one of the control electrodes and the test electrodes when applying at least one of a voltage and an electrical current to the control electrodes and/or the test electrodes, respectively. The system response for the control electrodes and the test electrodes may be measured separately. As an example, the impedance measurement unit may be configured for applying a current and/or a voltage, specifically an electrical potential, to at least one of the test electrodes of the test element using the contacts for electrically contacting the test element to perform the impedance test measurement. The impedance measurement unit may further be configured for applying a current and/or a voltage, specifically an electrical potential, to at least one of the control electrodes of the test element using the contacts for electrically contacting the test element to perform the impedance control measurement. The current may specifically be an alternating current. The voltage may specifically be an alternating voltage. Thus, the impedance of the sample of bodily fluid in the test region and in the control region may be measured using the impedance measurement unit, e.g. by applying an appropriate alternating voltage to the test electrodes and the control electrodes, respectively.

With regard to the term “impedance”, it shall be noted that in the context of the present disclosure the term is used interchangeably with the term “electrical impedance”. The term “impedance” as used herein is a broad term, and is to be given its ordinary and customary meaning to a person of ordinary skill in the art, and is not to be limited to a special or customized meaning. The term specifically may refer, without limitation, to at least one of: an electrical resistance; a complex electrical resistance; a measure of an opposition that an electronic circuit presents to a current when an alternating voltage is applied. Thus, the physical quantity of impedance may be regarded as extending the concept of resistance to alternating current circuits as the person of ordinary skill in the art will realize. The impedance of an electronic circuit may in particular indicate the ratio of the electrical voltage to the current, specifically given as a complex ratio, or a ratio having an amplitude and a phase.

The impedance test system may further have at least one evaluation unit, the evaluation unit being configured for determining the fibrinogen level by using measurement results from the electrical impedance test measurement and from the electrical impedance control measurement. The term “evaluation unit” as used herein is a broad term, and is to be given its ordinary and customary meaning to a person of ordinary skill in the art, and is not to be limited to a special or customized meaning. The term specifically may refer, without limitation, to an arbitrary functional element configured for analyzing and/or processing data. The evaluation unit may specifically analyze and/or process measurement data, e.g. the measurement results as generated by the impedance measurement unit. The evaluation unit may in particular comprise at least one processor. The processor may specifically be configured, such as by software programming, for performing one or more evaluation operations on the measurement results. The term “processor” as used herein is a broad term and is to be given its ordinary and customary meaning to a person of ordinary skill in the art and is not to be limited to a special or customized meaning. The term specifically may refer, without limitation, to an arbitrary logic circuitry configured for performing basic operations of a computer or system, and/or, generally, to a device which is configured for performing calculations or logic operations. In particular, the processor may be configured for processing basic instructions that drive the computer or system. As an example, the processor may comprise at least one arithmetic logic unit (ALU), at least one floating-point unit (FPU), such as a math co-processor or a numeric co-processor, a plurality of registers, specifically registers configured for supplying operands to the ALU and storing results of operations, and a memory, such as an L1 and L2 cache memory. In particular, the processor may be a multi-core processor. Specifically, the processor may be or may comprise a central processing unit (CPU). Additionally or alternatively, the processor may be or may comprise a microprocessor, thus specifically the processor's elements may be contained in one single integrated circuitry (IC) chip. Additionally or alternatively, the processor may be or may comprise one or more application-specific integrated circuits (ASICs) and/or one or more field-programmable gate arrays (FPGAs) and/or one or more tensor processing unit (TPU) and/or one or more chip, such as a dedicated machine learning optimized chip, or the like. The processor specifically may be configured, such as by software programming, for performing one or more evaluation operations. The evaluation unit may specifically be configured for determining the fibrinogen level by using the result of the electrical impedance control measurement as a basal impedance for taking into account at least one specific sample property of the bodily fluid other than the fibrinogen level, specifically a hematocrit value. The evaluation unit may for example, subtract the electrical impedance control measurement as a basal impedance from the electrical impedance control measurement, as will be discussed in detail further below. Other options of analyzing and/or processing the measurement data, specifically the electrical impedance control measurement and the electrical impedance test measurement are feasible.

The evaluation unit may further be configured for determining the fibrinogen level by using a predetermined or determinable relation between the measurement results of the electrical impedance test measurement and the electrical impedance control measurement and the fibrinogen level. In particular, the result of the impedance test measurement may depend on the fibrinogen level of the sample of bodily fluid. Thus, the detector compound present in the test region may specifically cleave fibrinogen thereby producing fibrin, whose clot formation may affect the impedance measurement in the test region but not in the control region, where fibrinogen remains uncleaved. The relation between the measurement results of the electrical impedance test measurement and the electrical impedance control measurement and the fibrinogen level may specifically be determined in a calibration process. The calibration process may for example be based on a plurality of samples of bodily fluid with different, known fibrinogen levels. The electrical impedance of each of the samples of bodily fluid may be determined in both the test region using the test electrodes and the control region using the control electrodes. The relation, as an example, may be programmed into the evaluation unit, wherein one or more measurement results from the electrical impedance test measurement and from the electrical impedance control measurement are used as an input and wherein the relation, specifically an algorithm programmed into the evaluation unit, derives, from the one or more measurement results, the at least one fibrinogen level. The relation, as an example, may be an analytical relation, such as a relation being programmed as an equation, wherein parameters of the equation are determined, e.g. by calibration measurements as outlined above. Additionally or alternatively, the relation, as an example, may be programmed as a lookup list or lookup table, wherein fibrinogen levels are given for various measurement results. Various options are feasible and generally known to the skilled person.

As an example, the relation may be a relation, e.g. a linear relation, between an impedance difference value ΔZ, being determined by subtracting a magnitude |Z| of an impedance value in the control region from a magnitude |Z| of an impedance value in the test region, and the time t of the measurement, e.g. a time t after sample application to the test element and/or to the test region and/or to the control region. As an example and as will be shown by exemplary embodiments below, the difference value ΔZ may be, at least in a determinable time interval, a linear function of the measurement time. The slope of this linear relation between the difference value ΔZ and the time t generally may depend on the fibrinogen level in the sample, so by experimentally determining the slope, e.g. by linear regression or fitting, the fibrinogen level may be determined. The fibrinogen level, as an example, may be a function of the slope which, as an example, may be determined by calibration measurements. As an example, the slope of the AZ vs. t-curves may be a linear function of the fibrinogen level, so the relation between the measurement results of the electrical impedance test measurement and the electrical impedance control measurement and the fibrinogen level may also be a multi-step relation, which may imply a first relation between the measurement results of the electrical impedance test measurement and the electrical impedance control measurement and at least one measurement parameter such as measurement time and/or measurement frequency, and a second relation between a parameter determined or determinable from the first relation and the fibrinogen level. Thus, generally, the determining of the fibrinogen level by using a predetermined or determinable relation between the measurement results of the electrical impedance test measurement and the electrical impedance control measurement and the fibrinogen level may imply using a one-step or multiple-step relation, specifically a linear relation, between an impedance difference value ΔZ, being determined by subtracting a magnitude |Z| of an impedance value in the control region from a magnitude |Z| of an impedance value in the test region, and the time t of the measurement or another measurement parameter such as the measurement frequency. Therein, at least one determinable parameter of this relation, e.g. a slope, may be a function of the fibrinogen level which may be determined by calibration. Other relations, however, are also feasible and may be used for determining the fibrinogen level from the measurement results of the at least one electrical impedance test measurement and the at least one electrical impedance control measurement.

The impedance test system may further be configured for determining a wetting of at least one of the control electrodes and the test electrodes. The impedance test system may further be configured for performing the electrical impedance test measurement and the electrical impedance control measurement at at least one predetermined point in time after the wetting. Thus, the impedance test system may be configured for detecting an application of the sample of bodily fluid to the test element, more specifically the presence of the sample in the test region and the control region. For detecting the wetting, again, one or more electrical measurements may be used, since wetting typically leads to changes in electrical resistances and/or electrical impedance. Thus, as an example, one or more of an electrical voltage, and electrical current or an electrical impedance may be monitored over the test electrodes and/or the control electrodes, wherein a rapid change in voltage, current or impedance, as an example, may be detected and interpreted as a wetting. When detecting a wetting, a timer may be started for the control measurement and/or the test measurement.

In a further aspect of the present disclosure, an impedance method of ex-vivo detecting, specifically in-vitro detecting, a fibrinogen level in a sample of a bodily fluid is disclosed. The method comprises the following steps, which, specifically, may be performed in the given order. It shall be noted, however, that a different order may also be possible. Further, one or more of the method steps may be performed once or repeatedly. Further, it may be possible to perform two or more of the method steps simultaneously or in a timely overlapping fashion. The method may comprise further method steps, which are not listed. In some aspects, the method steps comprise:
providing at least one test element as described herein, such as in any of the embodiments described above and/or as in any one of the embodiments described in further detail below;
applying the sample of the bodily fluid to at least one application opening of the capillary;
performing at least one electrical impedance test measurement by using the test electrodes;
performing at least one electrical impedance control measurement using the control electrodes; and
determining the fibrinogen level by using measurement results from the electrical impedance test measurement and from the electrical impedance control measurement.

The test element provided in step i) of the impedance method of ex-vivo detecting the fibrinogen level in a sample of a bodily fluid is a test element according to the present disclosure. Thus, regarding the test element, its definitions and embodiments, reference is made to the test description of the test element above and as described in further detail below. With respect to further terms, definitions and embodiments referred to in the context of the impedance method of ex-vivo detecting a fibrinogen level in a sample of a bodily fluid, reference is made to the description of the test element and the impedance test system above and as described in further detail below. The impedance method of ex-vivo detecting a fibrinogen level in a sample of a bodily fluid may in particular be performed using the impedance test system according to the present disclosure as described herein, such as in any of the embodiments described above and/or as in any one of the embodiments described in further detail below.

For possible embodiments of step v), reference may be made to the description of the evaluation unit above. Thus, specifically, for determining the fibrinogen level, the at least one relation may be used as described above. The at least one relation, as an example, may use one or more measurement results from the electrical impedance test measurement and from the electrical impedance control measurement in steps iii) and iv) as an input and may derive, from the one or more measurement results, the at least one fibrinogen level. The relation, as an example, may be or may comprise a computer-implemented algorithm. Thus, specifically, at least step v. may fully or partially be computer-implemented or at least computer-controlled. Additionally or alternatively, one or both of steps iii) and iv) may be computer-implemented and/or computer-controlled. Thus, at least one of steps iii)-v) may be computer-implemented, e.g. by providing appropriate instructions in the evaluation unit, specifically by programming at least one processor of the evaluation unit, in order to perform at least one of steps iii)-v).

The determining of the fibrinogen level may comprise at least one evaluation of the electrical impedance test measurement and the electrical impedance control measurement. The evaluation may depend on a configuration of the test element used in the impedance method, e.g. depending on a coating of the test electrodes and/or control electrodes, and/or on a property of the sample of the bodily fluid, e.g. depending on a presence and/or a concentration of hematocrit in the sample of the bodily fluid. The evaluation, as an example, may comprise determining at least one AZ vs. t-curve, as described above. The evaluation may comprise determining a minimum in the AZ vs. t-curve and analyzing a linear regime following the minimum in the AZ vs. t-curve. The linear regime may, as an example, be analyzed for determining a slope of the linear regime. The linear regime may be limited by an absolute predetermined range and/or a relative absolute range, e.g. a part of AZ values following the minimum, such as 20% of the AZ values following the minimum in the AZ vs. t-curve. The slope in the linear regime may be determined using linear regression. The fibrinogen level may be determined using the slope of the linear regime, for example using a relation between the slope of the linear regime and the fibrinogen level in the sample of the bodily fluid, as outlined in detail above.

In a further aspect of the present disclosure, a resistance test system for detecting a fibrinogen level in a sample of a bodily fluid is disclosed. The term “resistance test system” as used herein is a broad term and is to be given its ordinary and customary meaning to a person of ordinary skill in the art and is not to be limited to a special or customized meaning. The term specifically may refer, without limitation, to an arbitrary test system configured for performing at least one resistance test and/or at least one resistance analysis, e.g. a resistance analysis of a sample. Specifically, the resistance test system may be configured for performing an equivalent series resistance (ESR) measurement.

In some aspects, the test system comprises:
at least one test element according to the present disclosure, such as according to any one of the embodiments disclosed above and/or according to any one of the embodiments disclosed in further detail below; and
at least one test device having contacts for electrically contacting the test element, the test device further having at least one voltage measurement unit, wherein the voltage measurement unit is configured for applying at least one voltage signal to the test electrodes and the control electrodes of the test element, wherein the voltage measurement unit is further configured for determining at least one test response signal of the test region, wherein the voltage measurement unit is further configured for determining at least one control response signal of the control region.

The test element comprised by the resistance test system is a test element according to the present disclosure. Thus, regarding the test element, its definitions and embodiments, reference is made to the test description of the test element above and as described in further detail below. With respect to further terms, definitions and embodiments referred to in the context of the resistance test system, reference is also made to the description of the test element or the impedance test system above and as described in further detail below.

The test device may be or may comprise a test device as defined above. The test device may specifically be a resistance test device. Alternatively or additionally, the test device may comprise at least one microcontroller. The microcontroller may comprise at least one integrated circuit comprising one or more CPUs, one or more memory devices and/or one or more programmable input and output terminals. The microcontroller may specifically be a mixed signal microcontroller.

The term “voltage measurement unit” as used herein is a broad term and is to be given its ordinary and customary meaning to a person of ordinary skill in the art and is not to be limited to a special or customized meaning. The term specifically may refer, without limitation, to an arbitrary measurement unit configured for performing or controlling at least one resistance measurement. The voltage measurement unit may be configured for controlling further functional elements that may take part in the resistance measurement. The voltage measurement unit, for example, may be or may comprise at least one voltage source being configured to generate a voltage signal. Alternatively or additionally, the voltage measurement unit may comprise at least one function generator, such as at least one function generator configured for generating at least one of a square wave signal and a sine wave signal. The voltage measurement unit may be configured for separately controlling the at least one voltage signal applied to the test electrodes and the control electrodes of the test element. Further, the voltage measurement unit may be configured for measuring a voltage of the test electrodes and/or the control electrodes, respectively. For example, the voltage measurement unit may be or may comprise at least one signal analyzer, at least one oscilloscope and/or at least one voltmeter. The voltage measurement unit may specifically be configured for measuring at least one system response of the control electrodes and/or the test electrodes, the system response comprising at least one voltage response signal measured at at least one of the control electrodes and the test electrodes when applying a voltage to the control electrodes and/or the test electrodes, respectively. The system response for the control electrodes and the test electrodes may be measured separately.

The term “voltage signal” as used herein is a broad term and is to be given its ordinary and customary meaning to a person of ordinary skill in the art and is not to be limited to a special or customized meaning. The term specifically may refer, without limitation, to an arbitrary excitation voltage. The voltage signal may comprise a continuous voltage signal and/or a non-continuous voltage signal. For example, the voltage signal may comprise a single voltage pulse. The voltage pulse may be applied for a duration of 1 ns to 1 ms, specifically of 1 μs to 500 μs, more specifically of 200 μs. Alternatively or additionally, the voltage signal may comprise at least one of a square wave signal, a voltage pulse and a sequence of voltage pulses. The voltage signal may have an amplitude in the range from 0.1 V to 10 V, specifically from 1 V to 3 V.

The term “response signal” as used herein is a broad term and is to be given its ordinary and customary meaning to a person of ordinary skill in the art and is not to be limited to a special or customized meaning. The term specifically may refer, without limitation, to a voltage change determined at measurement electrodes in response to an applied excitation voltage signal. Specifically, the response signal may be or may comprise at least one voltage response of a system or region electrically contacted by one or more measurement electrodes via which the excitation voltage signal is applied to the system or region. As used herein, the term “test response signal” specifically may refer, without limitations, to a response signal of the test region. The test response signal may be obtained via the test electrodes of the test region. As further used herein, the term “control response signal” specifically may refer, without limitation, to a response signal of the control region. The control response signal may be obtained via the control electrodes of the control region.

The resistance test system may further have at least one evaluation unit. The evaluation unit may be or may comprise at least one evaluation unit as defined above with respect to the impedance test system. The evaluation unit may be configured for determining the fibrinogen level by using the test response signal and the control response signal. Specifically, the evaluation unit may be configured, such as by software programming of at least one processor, for performing one or more evaluation operations for determining the fibrinogen level by using the test response signal and the control response signal. For example, the evaluation unit may be configured for performing at least steps iv) and v) of a resistance method of ex-vivo detecting, specifically in-vitro detecting, a fibrinogen level in a sample of a bodily fluid, as will be outlined in further detail below.

The resistance test system may further comprise at least one reference resistor. The term “reference resistor” as used herein is a broad term and is to be given its ordinary and customary meaning to a person of ordinary skill in the art and is not to be limited to a special or customized meaning. The term specifically may refer, without limitation, to a resistor having a predetermined or pre-defined resistance value. The reference resistance may be an average value determined, specifically pre-determined, from a plurality of reference measurements. The test device may be configured for electrically contacting the test element via the reference resistor. Specifically, the reference resistor may be connected, e.g. directly or by an electrical line, to at least one of the contacts of the test device. The reference resistor may be arranged in between the test device and the test element.

The resistance test system may further comprise at least one voltage divider. The voltage divider may comprise the reference resistor and one of the test region or the control region of the test element. Specifically, the resistance test system may comprise two reference resistors, each reference resistor forming with at least one of the test region and the control region one voltage divider. For example, the test device may comprise at least two contacts, e.g. two outputs, wherein a first contact may be connected to the test region, specifically to the test electrodes, via a first reference resistor and wherein a second contact may be connected to the control region, specifically to the control electrodes, via a second reference resistor. The voltage measurement unit may be adapted to apply the voltage signal to the reference resistor, specifically to both reference resistors. The reference resistor may be connected to at least one contact, specifically output contacts, of the voltage measurement unit. The reference resistor may be arranged in series with the voltage measurement unit.

The resistance test system may further be configured for determining a wetting of at least one of the control electrodes and the test electrodes. The resistance test system may further be configured for applying the at least one voltage signal to the test electrodes and the control electrodes of the test element at at least one predetermined point in time after the wetting.

In a further aspect, a resistance method of ex-vivo detecting, specifically in-vitro detecting, a fibrinogen level in a sample of a bodily fluid, is disclosed. The method comprises the following steps, which, specifically, may be performed in the given order. It shall be noted, however, that a different order may also be possible. Further, one or more of the method steps may be performed once or repeatedly. Further, it may be possible to perform two or more of the method steps simultaneously or in a timely overlapping fashion. The method may comprise further method steps, which are not listed.

The method may comprise one or more (e.g., any combination) of the following steps:
providing at least one test element according to the present disclosure, such as according to any one of the embodiments disclosed above and/or according to any one of the embodiments disclosed in further detail below;
applying the sample of the bodily fluid to at least one application opening of the capillary;
applying at least one voltage signal to the test electrodes and the control electrodes of the test element;
determining at least one test response signal of the test region and at least one control response signal of the control region; and
determining the fibrinogen level by using the test response signal and the control response signal.

In the method, the resistance test system according to the present disclosure, such as according to any one of the embodiments disclosed above and/or according to any one of the embodiments disclosed in further detail below, may be used. Thus, regarding the test element and/or the resistance test system, definitions and embodiments thereof, reference is made to the test description of the test element and of the resistance test system above.

The voltage signal may comprise at least one of: a square wave signal; a voltage pulse; a sequence of voltage pulses.

For example, the voltage signal may comprise a sequence of voltage pulses. Each voltage pulse may be applied for a pulse duration of 1 ns to 1 ms, specifically of 1 μs to 500 μs, more specifically of 200 μs. Each voltage pulse may have a pulse amplitude in the range from 0.1 V to 10 V, specifically from 1 V to 3 V. The voltage pulses of the sequence of voltage pulses may specifically comprise equal pulse amplitude and/or pulse duration. In step iv), a plurality of test response signals of the test region and a plurality of control response signals of the control region may be determined for each voltage pulse of the sequence of voltage pulses. Step v. may comprise determining a minimum of a difference between the plurality of test response signals and the plurality of control response signals for each voltage pulse. Further, the method may comprise averaging the minima of the difference between the plurality of test response signals and the plurality of control response signals over the sequence of voltage pulses.

Steps iii) and iv) may be performed repeatedly during at least one measurement duration. Specifically, the sequence of voltage pulses may be applied multiple times within the measurement duration. Steps iii. and iv. may specifically be performed multiple times in regular time intervals within the measurement duration. For example, the method may comprise applying a sequence of a number of 200 voltage pulses, each voltage pulse being applied for 200 μs, wherein the sequence of voltage pulses is applied within a time interval of 100 ms. However, other numbers and/or duration of the voltage pulses are also feasible. The measurement duration may be in the range from 30 s to 1000 s, specifically from 100 s to 500 s, more specifically may be 180 s. The sequence of voltage pulses may be applied for every time interval of 100 ms within the measurement duration.

Further, the method may comprise determining a plurality of averaged minima of the difference between the plurality of test response signals and the plurality of control response signals within the measurement duration as a function of time. Specifically, for each sequence of voltage pulses, at least one averaged minimum of the difference between the plurality of test response signals and the plurality of control response signals may be determined. Applying the sequence of voltage pulses a multiple times within the measurement duration may yield the plurality of averaged minima of the difference between the plurality of test response signals and the plurality of control response signals.

The method may further comprise determining at least one slope of a linear regime evolving from a minimum of the plurality of averaged minima of the difference between the plurality of test response signals and the plurality of control response signals. The linear regime may be limited by at least one predetermined range following the minimum of the plurality of averaged minima of the difference between the plurality of test response signals and the plurality of control response signals. The predetermined range may comprise an absolute predetermined range or a relative predetermined range, specifically a predetermined range comprising a part of the averaged minima following the minimum of the plurality of averaged minima. For example, the predetermined range may comprise a number of 25% of the averaged minima starting from the minimum of the plurality of averaged minima to the end of the measurement duration. Further, the fibrinogen level may be determined by using the slope of the linear regime. Specifically, the fibrinogen level may be determined using at least one relation relating the slope of the linear regime to the fibrinogen level. The relation, as an example, may be or may comprise at least one analytical relation, such as a relation being programmed as an equation, wherein parameters of the equation are determined, e.g. by calibration measurements. Additionally or alternatively, the relation, as an example, may be programmed as a lookup list or lookup table, wherein fibrinogen levels are given for various measurement results, such as for various slopes of the linear regime. Various options are feasible and generally known to the skilled person.

Further, the voltage pulses of the sequence of voltage pulses may vary in pulse amplitude and/or pulse duration. For example, the sequence of voltage pulses may comprise a multi-pulse having two or more single pulses with different pulse amplitudes. The response signals may be evaluated with respect to the pulse amplitude, e.g. for determining a presence and/or a level of hematocrit in the sample of the bodily fluid. Alternatively or additionally, the test response signals of the test region and/or the control response signals of the control region may be averaged relatively to the pulse amplitude of the multi-pulse excitation pulse. This may allow for noise-cancelling in the response signals and, thus, for improving a signal-to-noise ratio.

The resistance method may allow for a fast ex-vivo detecting of the fibrinogen level in the sample of the bodily fluid. The resistance method may comprise, as an example, applying the sequence of voltage pulses, e.g. applying a number of at least 100, specifically at least 200, voltage pulses during a time interval within the measurement duration. The resistance measurement may allow for averaging the response signals on the fly and, thus, for evaluating averaged response signals.

Alternatively or additionally, the method may comprise determining a wetting of at least one of the control electrodes and the test electrodes. Steps iii. to v. may be performed at at least one predetermined point in time after determination of the wetting. The determining of the wetting may specifically be performed prior to step iii.

Alternatively or additionally, the method may further comprise controlling at least one temperature of at least one of the test region and the control region of the test element. The controlling of the temperature may specifically be performed at least once before and/or during method steps i. to v.

In a further aspect of the present disclosure, a computer program is disclosed, comprising instructions which, when the program is executed by a resistance test system according to the present disclosure, such as according to any one of the embodiments described above and/or according to any one of the embodiments disclosed in further detail below, cause the resistance test system to perform the resistance method of ex-vivo detecting a fibrinogen level in a sample of a bodily fluid according to the present disclosure, such as according to any one of the embodiments described above and/or according to any one of the embodiments disclosed in further detail below.

In a further aspect of the present disclosure, a computer-readable storage medium, specifically a non-transient computer readable storage medium, is disclosed, comprising instructions which, when the instructions are executed by a resistance test system according to the present disclosure, such as according to any one of the embodiments described above and/or according to any one of the embodiments disclosed in further detail below, cause the resistance test system to perform the resistance method of ex-vivo detecting a fibrinogen level in a sample of a bodily fluid according to the present disclosure, such as according to any one of the embodiments described above and/or according to any one of the embodiments disclosed in further detail below.

As used herein, the term “computer-readable storage medium” specifically may refer to non-transitory data storage means, such as a hardware storage medium having stored thereon computer-executable instructions. The computer-readable storage medium specifically may be or may comprise a storage medium such as a random-access memory (RAM) and/or a read-only memory (ROM). The computer-readable storage medium specifically may be or may comprise a computer-readable data carrier.

Thus, specifically, one, more than one or even all of method steps iii) to v) of the resistance method as indicated above may be performed by using a computer or a computer network, preferably by using a computer program.

Further disclosed and proposed herein is a computer program product having program code means, in order to perform the resistance method according to the present disclosure in one or more of the embodiments enclosed herein, specifically method steps iii) to v) of the resistance method, when the program is executed on a computer or computer network. Specifically, the program code means may be stored on a computer-readable data carrier and/or on a computer-readable storage medium.

Further disclosed and proposed herein is a data carrier having a data structure stored thereon, which, after loading into a computer or computer network, such as into a working memory or main memory of the computer or computer network, may execute the resistance method according to one or more of the embodiments disclosed herein, specifically method steps iii) to v) of the resistance method.

Further disclosed and proposed herein is a non-transient computer-readable medium including instructions that, when executed by one or more processors, cause the one or more processors to perform the resistance method according to one or more of the embodiments disclosed herein, specifically method steps iii) to v) of the resistance method.

Further disclosed and proposed herein is a computer program product with program code means stored on a machine-readable carrier, in order to perform the resistance method according to one or more of the embodiments disclosed herein, specifically method steps iii) to v) of the resistance method, when the program is executed on a computer or computer network. As used herein, a computer program product refers to the program as a tradable product. The product may generally exist in an arbitrary format, such as in a paper format, or on a computer-readable data carrier and/or on a computer-readable storage medium. Specifically, the computer program product may be distributed over a data network.

Finally, disclosed and proposed herein is a modulated data signal which contains instructions readable by a computer system or computer network, for performing the resistance method according to one or more of the embodiments disclosed herein, specifically method steps iii) to v) of the resistance method.

Referring to the computer-implemented aspects of the disclosure, one or more of the method steps or even all of the method steps of the resistance method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements. Specifically, further disclosed herein are:
a computer or computer network comprising at least one processor, wherein the processor is adapted to perform the resistance method according to one of the embodiments described in this description, specifically method steps iii) to v) of the resistance method,
a computer loadable data structure that is adapted to perform the resistance method according to one of the embodiments described in this description, specifically method steps iii) to v) of the resistance method, while the data structure is being executed on a computer,
a computer program, wherein the computer program is adapted to perform the resistance method according to one of the embodiments described in this description, specifically method steps iii) to v) of the resistance method, while the program is being executed on a computer,
a computer program comprising program means for performing the resistance method according to one of the embodiments described in this description, method steps iii) to v) of the resistance method, while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the resistance method according to one of the embodiments described in this description, specifically method steps iii) to v) of the resistance method, after having been loaded into a main and/or working storage of a computer or of a computer network, and
a computer program product having program code means, wherein the program code means can be stored or are stored on a storage medium, for performing the resistance method according to one of the embodiments described in this description, specifically method steps iii) to v) of the resistance method, if the program code means are executed on a computer or on a computer network.

In a further aspect of the present disclosure, a method of manufacturing a test element for detecting a fibrinogen level in a sample of a bodily fluid is disclosed. The method comprises the following steps, which, specifically, may be performed in the given order. It shall be noted, however, that a different order may also be possible. Further, one or more of the method steps may be performed once or repeatedly. Further, it may be possible to perform two or more of the method steps simultaneously or in a timely overlapping fashion. The method may comprise further method steps, which are not listed.

In some aspects, the method steps comprise:
i) providing at least one substrate having disposed thereon at least two test electrodes and at least two control electrodes;
ii) disposing at least one detector compound being capable of specifically cleaving and optionally activating fibrinogen;
iii) providing at least one spacer layer;
iv) providing at least one cover layer; and
v) forming a sandwich setup comprising, in the given order, the substrate, the spacer layer and the cover layer, wherein a capillary is provided in the sandwich setup having sidewalls formed by the spacer layer, with the test electrodes and the control electrodes facing the capillary, wherein at least one test region and at least one control region are formed within the capillary, wherein the test electrodes are disposed within the test region, wherein the control electrodes are disposed within the control region, and wherein the detector compound is disposed within the test region.

In step II), said detector compound may particularly be deposited by using a solution of the detector compound. Thus, the detector compound may upon application be dissolved in a liquid or paste-like solution. The solution may, however, as part of the manufacturing process dry, harden or solidify such that the detector compound may in the finished test element be present in the test region as a dried film or as part of the hardened detector compound layer. The at least one detector compound is capable of specifically cleaving fibrinogen. The detector compound may optionally be capable of activating fibrinogen. The detector compound may in particular be a serine endopeptidase (E.C.3.4.21).

The test element manufactured with the method of manufacturing a test element for detecting a fibrinogen level in a sample of a bodily fluid may specifically be the test element according to the present disclosure, e.g. according to any one of the embodiments described above and/or according to any one of the embodiments disclosed in further detail below. Thus, regarding the test element, its definitions and embodiments, reference is made to the description of the test element above and as described in further detail below. With respect to further terms, definitions and embodiments referred to in the context of the method of manufacturing a test element for detecting a fibrinogen level in a sample of a bodily fluid, reference is made to the description of the test element, the test systems and the methods of ex-vivo detecting, specifically in-vitro detecting, a fibrinogen level in a sample of a bodily fluid as given above and as described in further detail below.

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of the disclosure is not restricted by the preferred embodiments. The embodiments are schematically depicted in the figures. Therein, identical reference numbers in these figures refer to identical or functionally comparable elements.

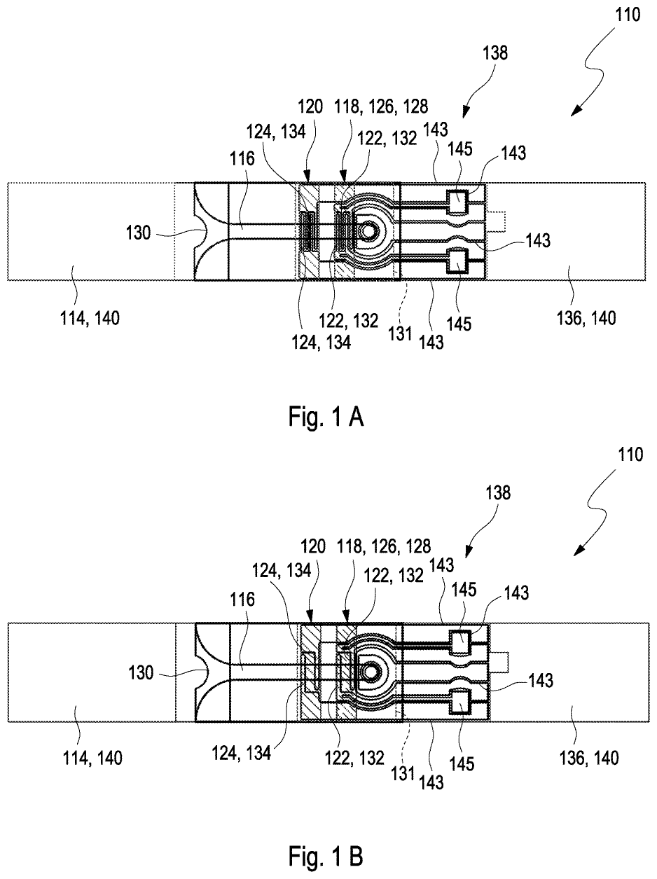

FIG. 1A shows a schematic top view of an embodiment of a test element 110 for detecting a fibrinogen level in a sample 112 of a bodily fluid according to the present disclosure. The test element 110 comprises at least one substrate 114 and at least one capillary 116 for receiving and transporting the sample 112. As indicated in the FIG. 1A, the test element 110 further comprises at least one test region 118 and at least one control region 120 within the capillary 116. The test element 110 comprises at least two test electrodes 122 in the test region 118, and at least two control electrodes 124 in the control region 120, as shown in FIG. 1A. The test element 110 further comprises, in the test region 118, at least one detector compound 126, the detector compound 126 being capable of specifically cleaving fibrinogen. The detector compound 126 may be comprised in at least one detector compound layer 128, as shown in FIG. 1A. In particular, the detector compound layer 128 may be disposed on at least one side wall of the capillary 116, extending over the entire length of the test region 118 in a direction of longitudinal extension of the capillary 116. Additionally or alternatively, the detector compound 126 may be disposed on the test electrodes 122 (not shown in the figures). Further, the test element 110 may in particular be devoid of the at least one detector compound 126 in the control region 120 as also illustrated in FIG. 1A.

The test region 118 and the control region 120 may be disposed in the same capillary 116, wherein the control electrodes 124 and the test electrodes 122 may be disposed within the capillary 116 such that the sample 112 of bodily fluid entering the capillary 116 may pass the control electrodes 124 before passing the test electrodes 122. This set-up is shown in the embodiment of the test element 110 displayed in FIG. 1A. In the case that the test region 118 and the control region 120 are disposed in the same capillary 116, the control electrodes 124 and the test electrodes 122 may in particular be spaced apart by a minimum distance of 1.0 mm to 2.5 mm, specifically by a minimum distance of 1.6 mm to 2.0 mm. Within typical reaction times, this separation may specifically be suited for avoiding a cross-talk from a detection reaction in the test region 118 into the control region 120, such that a control measurement in the control region 120 may remain unaffected by a reaction in the test region 118.

Alternatively, the test element 110 may comprise at least two capillaries 116, wherein the test region 118 and the control region 120 may be disposed in different capillaries 116, specifically in different capillaries 116 having a common application opening 130. In this set-up, which is not shown in the present figures, a minimum distance between the common application opening 130 of the capillaries 116 and the control electrodes 124 and a minimum distance between the common application opening 130 and the test electrodes 122 may be essentially identical.

As illustrated in FIG. 1A, the test element 110 may specifically be a strip-shaped test element 110. The substrate 114 may in particular be a flexible substrate 114. The test element 110 may contain at least one application opening 130 of the capillary 116, as shown in FIG. 1A. The test element 110 may further contain at least one vent opening of the capillary 116, wherein the test region 118 and the control region 120 are disposed in between the application opening 130 and the vent opening (not shown in the figures). The test electrodes 122 and the control electrodes 124 may be disposed on the substrate 114, specifically on the same side of the same substrate 114, as indicated in FIG. 1A. Further, the test region 118 and the control region 120 may have essentially identical dimensions as can also be seen in FIG. 1A. As further displayed in FIG. 1A, the test electrodes 122 and the control electrodes 124 may have an essentially identical geometry. The test electrodes 122 may contain test electrode fingers 132 spaced apart in a direction of longitudinal extension of the capillary 116, as shown in FIG. 1A. The test electrode fingers 132 may in particular be spaced apart by 0.10 mm to 1.00 mm, specifically by 0.20 mm to 0.80 mm. The test electrode fingers 132 may particularly have a width of 0.1 mm to 0.5 mm, specifically of 0.25 mm to 0.4 mm. The test electrodes 122 may specifically comprise two test electrodes 122, the two test electrodes 122 having interleaving test electrode fingers 132 as shown in FIG. 1A. As further indicated in FIG. 1A, the control electrodes 124 may contain control electrode fingers 134 spaced apart in a direction of longitudinal extension of the capillary 116. The control electrode fingers 134 may specifically be spaced apart by 0.10 mm to 1.00 mm, specifically by 0.20 mm to 0.80 mm. In particular, the control electrode fingers 134 may have a width of 0.1 mm to 0.5 mm, specifically of 0.25 mm to 0.4 mm. The control electrodes 124 may specifically comprise two control electrodes 124, the two control electrodes 124 having interleaving control electrode fingers 134 as shown in FIG. 1A. As will be shown in further detail below, these dimensions and/or geometries specifically are suited for performing impedance measurements in samples 112 of blood and/or interstitial fluid, generating a sufficient signals and sufficient signal-to-noise ratio.

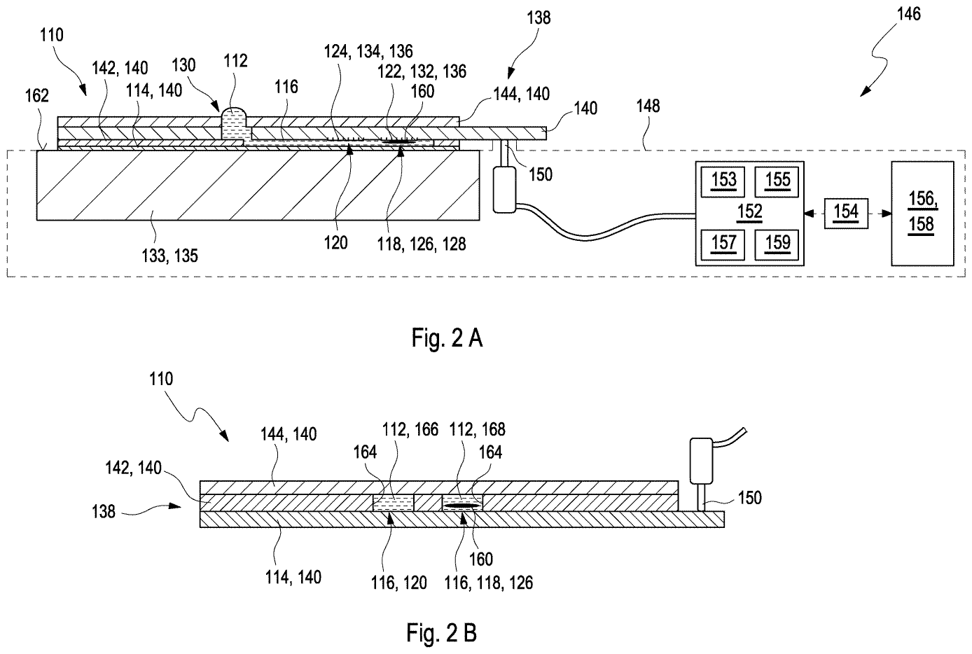

The test electrodes 122 and the control electrodes 124 may, in particular, be formed using an electrode foil 136 as illustrated in FIG. 1A. The electrode foil 136 may be part of a layer composition 138, also referred to as a sandwich setup, of the test element 110. The test electrodes 122 and the control electrodes 124 may, e.g. be formed as cutouts of a layer 140, e.g. an insulating layer, covering the electrode foil 136. Alternatively, the test electrodes 122 and the control electrodes 124 may be applied directly on the substrate 114, e.g. by printing the electrodes on the substrate 114 and/or by structuring the electrodes by laser ablation from a substrate foil which is coated by a thin layer having a thickness between 10 and 200 nm, e.g. a thin layer of gold. The layer composition 138 may comprise a plurality of layers 140 as shown in the cross-sectional view of the test element 110 in FIG. 2A. The layer composition 138 may for example comprise the substrate 114, the electrode foil 136, at least one spacer layer 142, at least one cover layer 144. The layer composition 138 may comprise at least one further layer 140. The layers 140 may cover each other completely or only partially, as indicated in FIG. 1A, where the electrode foil 136 partially covers the substrate 114. The cover layer 144 is not shown in FIGS. 1A-1B. Instead, FIGS. 1A-1B both allow a top view on an embodiment of the test element 110 with removed cover layer 144 for a more detailed view on a plurality of functional elements of the test element 110, such as the test electrodes 122, the control electrodes 124, the capillary 116 and further elements as described above and as further described below.

The test electrodes 122 and the control electrodes 124 may comprise contact leads 143 having contact pads 145. The contact pads of the test electrodes 122 and the control electrodes 124 may particularly be disposed on the same side of the substrate 114 as shown in FIG. 1A. The electrodes 122, 124 may all be electrically contacted by separate contact leads 143. Alternatively, however, two or more of the electrodes 122, 124 may also be contacted by at least one common contact lead 143. This option is not shown in the figures. Specifically, at least a first one of the test electrodes 122 and at least a first one of the control electrodes 124 may be electrically contacted via a common contact lead 143, wherein at least a second one of the test electrodes 122 and at least a second one of the control electrodes 124 may be electrically contacted via separate contact leads 143. Thereby, the number of electrical contact leads 143 may be reduced, which specifically may save space and costs.

The test element 110 comprises the capillary 116 as shown in FIG. 1A. The capillary 116 may in particular have a width of 0.5 mm to 2.5 mm, specifically of 1.0 to 2.0 mm, more specifically of 1.5 mm. Specifically, the capillary 116 may have a height of 50 μm to 500 μm, specifically of 100 μm to 300 μm, more specifically of 175 μm. These dimensions are suited, specifically, for conducting bodily fluids such as blood and/or interstitial fluid by capillary forces.

The test element 110 may further comprise a heating zone 131. As can be seen in FIG. 1A, the heating zone 131 may in particular comprise the control region 120 and the test region 118. Thus, a temperature in the heating zone 131, specifically in the control region 120 and the test region 118, may be adjustable and/or controllable e.g. by positioning the test element 110 on at least one heating element 133. The heating element 133 may in particular be integrated in a test element receptacle 135 as shown in FIG. 2A.

So far, FIG. 1A has been described in detail. FIG. 1B shows a further embodiment of the test element 110 according to the present disclosure. The top views of the embodiments shown in FIG. 2. 1A-1B are essentially identical except for an electrode design of the test electrodes 122 and the control electrodes 124. Thus, with regard to FIG. 1B attention shall focus on the electrode design while reference is made to the description of FIG. 1A regarding other aspects such as the substrate 114, the capillary 116, the test region 118 and the control region 120. Just as the test element 110 displayed in FIG. 1A, the test element 110 shown in FIG. 1B comprises one pair of test electrodes 122 with interleaving electrode fingers. While the two test electrodes 122 of the test element 110 in FIG. 1A form two test electrode fingers 132 each, the two test electrodes 122 of the test element 110 in FIG. 1B form one test electrode finger 132 each. The same is true for the two control electrodes 124, which in the test element 110 in FIG. 1A form two control electrode fingers 134 each, while the two control electrodes 122 of the test element 110 in FIG. 1B form just one control electrode finger 134 each. As will be shown in detail further below, the electrode design of the test electrodes 122 and the control electrodes 124 may affect the detection of the fibrinogen level by affecting measurements performed with the test electrodes 122 and the control electrodes 124.

FIG. 2A shows a schematic cross-sectional view of an embodiment of an impedance test system 146 for detecting a fibrinogen level in a sample 112 of a bodily fluid. The impedance test system 146 comprises at least one test element 110 as described herein, such as in any of the embodiments described above and/or as in any one of the embodiments described in further detail below. The impedance test system 146 further comprises at least one test device 148 having contacts 150 for electrically contacting the test element 110 as illustrated in FIG. 2A. The test device 148 further comprises at least one impedance measurement unit 152, wherein the impedance measurement unit 152 is configured for performing at least one electrical impedance test measurement using the test electrodes 122, wherein the impedance measurement unit 150 is further configured for performing at least one electrical impedance control measurement using the control electrodes 124.

The test device 148 and the test element 110 may be electrically connectable via the contacts 150 of the test device 148 as shown in FIG. 2A. Specifically, an electrical current and/or an electrical voltage, more specifically an electrical potential, may be applied to the test electrodes 122 and/or the control electrodes 124 of the test element 110 using the test device 148. The test device 148, specifically the impedance measurement unit 152, may particularly comprise a potentiostat 154, as shown in FIG. 2A. The potentiostat 154 may in particular be configured in a three-electrode configuration for keeping a potential of at least one working electrode constant with regard to a high impedance reference electrode by applying a suitable potential between the counter and the working electrode. In this example, the potentiostat 154 may be operated in a two-electrode configuration, wherein one electrode of the test electrodes 122 and the control electrodes 124 may be connected to the working electrode connector and the other electrode may be connected to the reference and counter electrode connector of the potentiostat 154.

In particular, the test element 110 may comprise at least one pair of test electrodes 122 and at least one pair of control electrodes 124. Specifically, one test electrode 122 of each pair of the test electrodes 122 and/or one control electrode 124 of each pair of control electrodes 124 may function as working electrodes, while the other electrode of each pair may function as an auxiliary electrode. Further, the potentiostat 154 may be configured for measuring at least one current and at least one voltage.

The test device 148, in particular the impedance measurement unit 152, may in particular comprise the at least one potentiostat 154 as shown in FIGS. 2A-2B. Additionally or alternatively, the test device 148, in particular the impedance measurement unit 152, may, in particular comprise at least one current source 153 and/or at least one voltage source 155, for applying at least one of a current and a voltage to the test electrodes 122 and/or the control electrodes 124, e.g. via the electrical contacts 150. The current source 153 may in particular supply the test element 110 with at least one alternating current. The voltage source 155 may in particular supply the test element 110 with at least one alternating voltage. Further, the test device 148, in particular the impedance measurement unit 152 as illustrated in FIG. 2A, may, in particular, comprise at least one of a voltage measurement device 159 and/or a current measurement device 157, specifically for measuring at least one of a current and a voltage of the test electrodes 122 and/or the control electrodes 124, respectively. Specifically, the test device 148, in particular the impedance measurement unit 152, may be configured for measuring at least one system response of the control electrodes 124 and/or the test electrodes 122, the system response being at least one of a current signal and a voltage signal measured at at least one of the control electrodes 124 and the test electrodes 122 when applying at least one of a voltage and an electrical current to the control electrodes 124 and/or the test electrodes 122, respectively. The system response for the control electrodes 124 and the test electrodes 122 may be measured separately. As an example, the impedance measurement unit 152 may be configured for applying a current and/or a voltage, specifically an electrical potential, to at least one of the test electrodes 122 of the test element 110 using the contacts 150 for electrically contacting the test element 110 to perform the impedance test measurement. The impedance measurement unit 152 may further be configured for applying a current and/or a voltage, specifically an electrical potential, to at least one of the control electrodes 124 of the test element 110 using the contacts 150 for electrically contacting the test element 110 to perform the impedance control measurement. The current may specifically be an alternating current. The voltage may specifically be an alternating voltage. Thus, the impedance of the sample 112 of bodily fluid in the test region 118 and in the control region 120 may be measured using the impedance measurement unit 152, e.g. by applying an appropriate alternating voltage to the test electrodes 122 and the control electrodes 124, respectively.

The impedance test system 146 may further have at least one evaluation unit 156, the evaluation unit 156 being configured for determining the fibrinogen level by using measurement results from the electrical impedance test measurement and from the electrical impedance control measurement. In particular, the evaluation unit 156 may determine the fibrinogen level of the sample 112 of bodily fluid by using measurement results generated by the impedance measurement unit 152. In FIG. 2A an exchange of data, which may comprise e.g. the measurement results generated by the impedance measurement unit 152, between the impedance measurement unit 152 and the evaluation unit 156 is indicated by the dashed arrow. The evaluation unit 156 may in particular comprise at least one processor 158 as shown in FIG. 2A. The processor 158 may specifically be configured for performing one or more evaluation operations on the measurement results. The evaluation unit 156 may further be configured for determining the fibrinogen level by using a predetermined relation between the measurement results of the electrical impedance test measurement and the electrical impedance control measurement and the fibrinogen level. In particular, the result of the impedance test measurement may depend on the fibrinogen level of the sample 112 of bodily fluid. Thus, the detector compound 126 present in the test region 118 may specifically cleave fibrinogen thereby producing fibrin, whose clot formation may affect the impedance measurement in the test region 118 but not in the control region 120, where fibrinogen remains uncleaved. FIG. 2A shows a fibrin clot 160 that has formed in the test region 118 due to the presence of the detector compound 126.

As indicated by the dashed line in FIG. 2A, the test device 148, in particular the impedance measurement unit 152, which may specifically comprise the potentiostat 154, may be constructionally combined with further functional elements, such as the impedance measurement unit 152, the evaluation unit 156, the test element receptacle 135, the heating element 133, and even further elements into a single device, e.g. into a portable and/or handheld device. Thus, a patient or user may be enabled to determine the fibrinogen levels of the sample of bodily fluid in a flexible and user-friendly manner, e.g. by inserting, applying or depositing the test element 110 into or onto the test element receptacle 135 respectively. The test element receptacle 135 may in particular comprise at least one support surface 162 for receiving the test element 110, as shown in FIG. 2A. Additionally or alternatively, the test element 110 may be received in a different manner. Thus, the test element receptacle 135 may comprise a slot, a flap or the like. Other options are feasible.

The impedance test system 146 shown in FIG. 2A comprises the test element 110. In the cross-sectional view of FIG. 2A the layer composition 138 of the embodiment of the test element 110 as schematically depicted in FIG. 2A can be seen. The test element 110 may in particular comprise at least one cover layer 144 and at least one spacer layer 142 disposed between the substrate 114 and the cover layer 144, wherein the spacer layer 142 may form sidewalls 164 of the capillary 116, as shown in FIG. 2A. As can also be seen in FIG. 2A, the test element 110 may comprise at least one further layer 140 that may be sandwiched between the just mentioned layers 140, such as the substrate 114, the spacer layer 142 and the cover layer 144. The further layer 140 may in particular be sandwiched between the spacer layer 142 and the cover layer 144, as shown in FIG. 2A. Other options are feasible.

While FIG. 2A shows a cross-sectional view of an embodiment of the impedance test system 146 comprising the test element 110, FIG. 2B shows a cross-sectional view of a further embodiment of the test element 110 in contact with the potentiostat 154. The test element 110 comprises the at least one capillary 116. The test region 118 and the control region 120 may be disposed in the same capillary 116, wherein the control electrodes 124 and the test electrodes 122 are disposed within the capillary 116 such that the sample 112 of bodily fluid entering the capillary 116 passes the control electrodes 124 before passing the test electrodes 122. This possible setup is shown in FIG. 2A. Alternatively, the test element 110 may comprise at least two capillaries 116, wherein the test region 118 and the control region 120 are disposed in different capillaries 116 as can be seen in FIG. 2B. The different capillaries 116 may specifically have a common application opening 130 (not shown in FIG. 2B). Further, a minimum distance between the common application opening 130 of the capillaries 116 and the control electrodes 124 and a minimum distance between the common application opening 130 and the test electrodes 122 may be essentially identical. Thus, the sample 112 of bodily fluid applied to the common application opening 130 may be split into at least two portions at a branching point of the capillary 116, wherein a first portion 166 of the sample 112 is directed to the control region 120, wherein a second portion 168 of the sample 112 is directed to the test region 118. The first portion 166 of the sample 112 and the second portion 168 of the sample 112 may have covered the same distance when they reach the control region 120 and the test region 118, respectively.

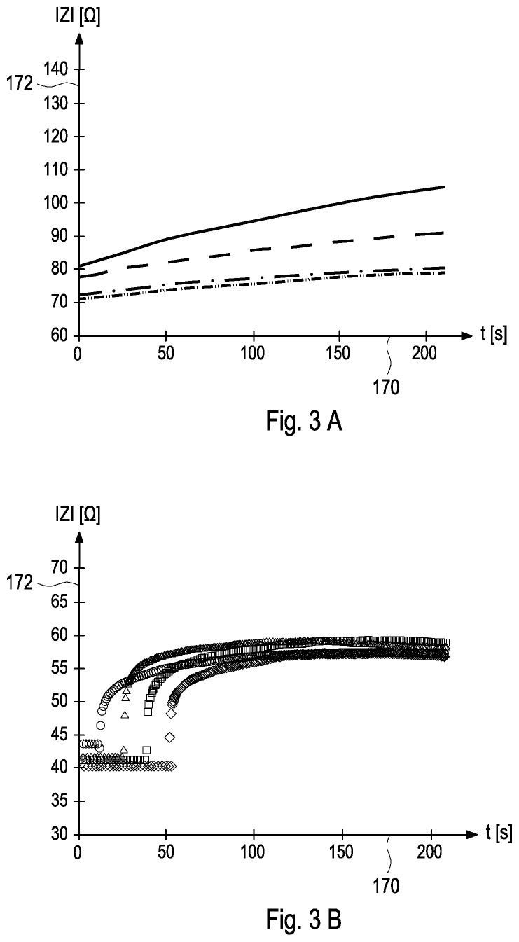

FIG. 3A shows a measurement diagram of an electrical impedance control measurement. FIG. 3B shows a measurement diagram of an electrical impedance test measurement. Each Figure shows a measurement diagram, wherein the time in seconds is plotted on the horizontal axis 170, while an absolute value |Z| of the complex number Z, which represents the impedance, is plotted on the vertical axis 172. The absolute value |Z| may also be referred to as the modulus or the magnitude of the complex number. The unit of the impedance plotted on the vertical axis 172 is ohm. Both measurement diagrams show data of an exemplary experiment with four different test elements 110, specifically being embodied identical with respect to each other. FIG. 3A specifically shows the electrical impedance measurements of the control regions 120 of the four test elements 110, wherein FIG. 3B shows the electrical impedance measurements of the test regions 118 of the four test elements 110. Further, both measurement diagrams show the course of |Z| over a time of about 200 seconds at an alternating voltage of 100 kHz. While the data displayed in FIG. 3A is acquired in the absence of the detector compound 126, the data displayed in FIG. 3B shows the effect of the addition of the detector compound 126 to the samples 112 of bodily fluid. All four test elements 110 were exposed to the same sample concentration of fibrinogen. The reactive substance on the test regions 118 was the same for each test element 110, in this example a volume of 0.5 μL of a Thrombin solution with a concentration of 0.1 Unit/μL. The same native whole blood sample was applied to each test element 110, in this example 4.5 μL of blood to the control region 120 and 4.5 μL of blood to the test region 118. A test stand with four measurement channels was equipped with the four identical test elements 110. The volume of the sample of whole blood of 4.5 μL was added each to the test regions 118 and the control regions 120 of all four test elements 110. Thus, all test elements 110 had a sample of 4.5 μL blood in each of the test region 118 and the control region 120. A volume of 0.5 μL of a Thrombin solution was added to each test region 118 of the four test elements 110 and the impedance measurements were started subsequently at a frequency of 100 kHz. The electrical impedance measurements were recorded at 100 kHz over time for each of the four test elements 110 simultaneously via the four measurement channels. The concentration of fibrinogen in the native whole blood was assumed to be in the normal range of Fibrinogen.

The addition 174 of the detector compound 126 takes place at different time points for the four test elements 110 and is indicated in FIG. 3B directly at the curve for each test element 110. As becomes obvious from the measurement diagram in FIG. 3B, the addition of the detector compound, which induces fibrin clot 160 formation, goes along with a sudden and significant increase in the impedance. The four test elements 110 in FIG. 3A, though showing a certain drift in their impedance over time, do not show sudden or drastic changes in their impedance. An amplitude of the alternating voltage for the impedance control and/or test measurement may be in the range of 1 to 30 mV RMS, specifically in the range of 5 to 15 mV RMS, more specifically 10 mV RMS. Therein, the root mean square (RMS) amplitude may be determined according to V_{RMS}=V_{PP}/\sqrt{2}, wherein Vₚₚ denotes the peak-to-peak amplitude of the alternating voltage. As can be seen in FIG. 3B, the measurement curves of the electrical impedance test measurements for the four test elements 110 overlap with each other. Thus, this experiment may show the reproducibility of the impedance method of ex-vivo detecting the fibrinogen level in the sample 112 of the bodily fluid, in this example in the sample of native whole blood.

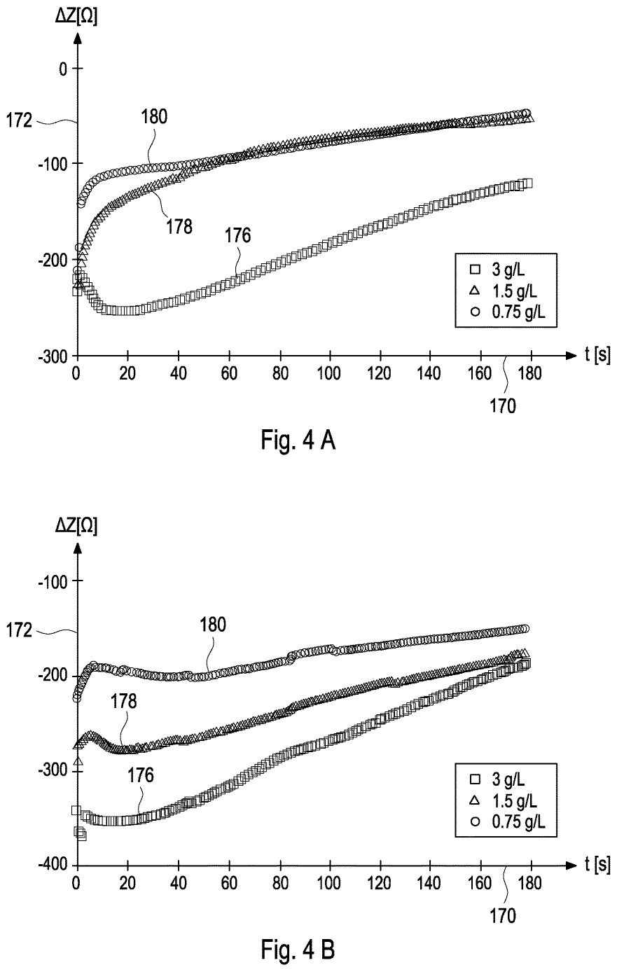

FIG. 4A shows a measurement diagram of an electrical impedance test measurement acquired at a voltage of 10 kHz. FIG. 4B shows a measurement diagram of an electrical impedance test measurement acquired at a voltage of 100 kHz. In both measurement diagrams the time in seconds is plotted on the horizontal axis 170. On the vertical axis 172, ΔZ is plotted in ohm. ΔZ is determined by subtracting from the magnitude |Z| as measured in the test region 118 the magnitude |Z| of a corresponding sample 112 measured in the control region 120. Corresponding samples may be identical samples 112 or samples 112 with the same fibrinogen level. Data of such corresponding samples 112 acquired at a voltage alternating at 100 kHz are displayed in FIG. 3A in an exemplary fashion. ΔZ may also be referred to as a normalized value of |Z|. The normalized value of |Z| may for example, take into account the drift of |Z| as particularly observable in FIG. 3A. Normalization may also account for other effects, such as sample properties, e.g. a hematocrit value, or further effects, such as time-dependent effects, e.g. drying effects regarding the sample 112. While the measurement data displayed in FIG. 4A are recorded at a voltage alternating at 10 kHz, the data displayed in FIG. 4B are recorded at a voltage alternating at 100 kHz. Both measurement diagrams show three samples 112 each, which differ in the fibrinogen level, specifically in the fibrinogen concentration. The samples marked 176 comprise fibrinogen at a concentration of about 3 gram/Liter henceforth abbreviated as g/L. The samples marked 178 comprise fibrinogen at a concentration of about 1.5 g/L. The samples marked 180 comprise fibrinogen at a concentration of about 0.75 g/L. All samples show a certain drift in their impedance over time. At a voltage alternating at 10 kHz, the sample 176 comprising fibrinogen at a concentration of about 3 g/L can be clearly distinguished from the other two samples 178 and 180, while the latter two are indistinguishable for the greater part of the course of the measurement data. At a voltage alternating at 100 kHz, all three samples 176, 178 and 180 can be clearly distinguished in their impedance response to the alternating voltage applied. In the experiments just described, 3 g/L may correspond to a normal fibrinogen level, 1.5 g/L may correspond to a medium fibrinogen level and 0.75 g/L may correspond to a low fibrinogen level.

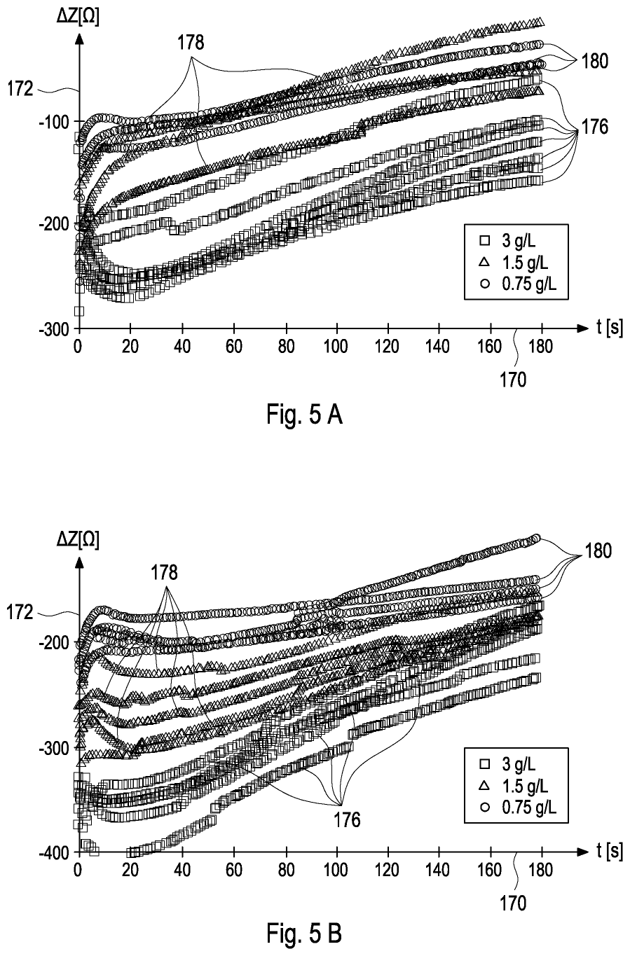

FIGS. 5A-B show the data displayed in FIGS. 4A-4B, respectively. In addition FIGS. 5A-5B show a number of replicates such that each fibrinogen level is represented with at least three samples 112. Again, the data indicate that at a voltage alternating at 100 kHz, the samples 112 may be distinguished in their impedance response to the alternating voltage according to the fibrinogen concentration they comprise. At a voltage alternating at 10 kHz, the sample 176 comprising fibrinogen at a concentration of about 3 g/L can be distinguished from the samples 178 and 180, while the latter two are indistinguishable for the greater part of the course of the measurement data.

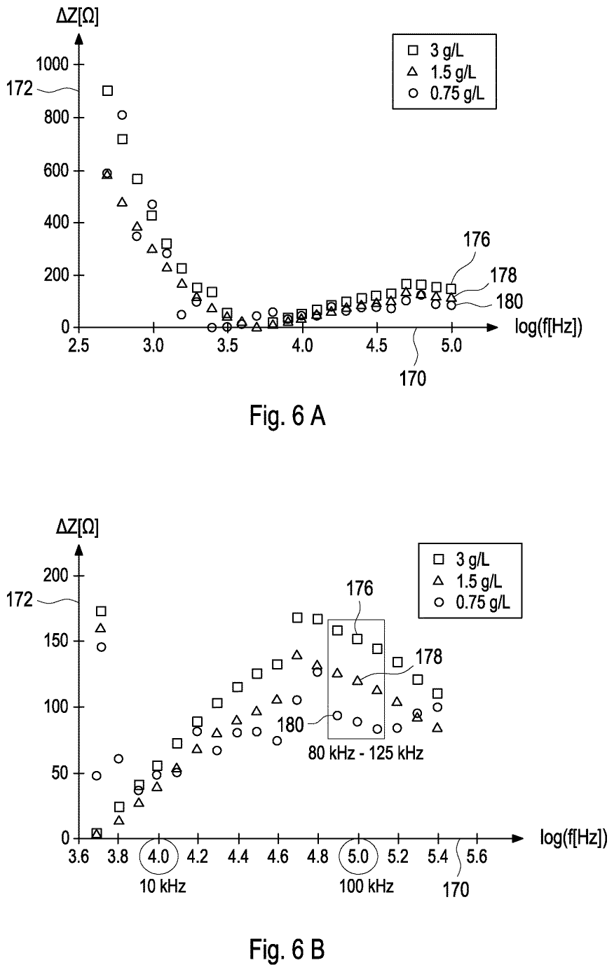

FIGS. 6A-6B each show a measurement diagram, also referred to as a Bode graph, of an impedance test measurement wherein the normalized impedance ΔZ [Ω] is plotted against the decadic logarithm of the frequency f [Hz] of the alternating voltage at which the impedance test measurement is carried out, denoted by log (f [Hz]). In both measurement diagrams the base 10 logarithm of the frequency of the alternating voltage is plotted on the horizontal axis 170, while ΔZ in ohm is plotted on the vertical axis 172. Measurement data acquired at voltages alternating at different frequencies are displayed. Each data point corresponds to the normalized impedance value ΔZ as determined from the impedance test measurement and the impedance control measurement at 180 seconds after sample application to the test element 110. FIG. 6B is an enlarged view of data also comprised by FIG. 6A as can easily be seen by a comparison of the respective horizontal and vertical axes of FIGS. 6A-6B. In both FIGS. 6A-6B data of three samples 112 are displayed, wherein the samples 112 vary in the fibrinogen level as described above. A first sample 112 comprises 3 g/L fibrinogen (176), a second sample comprises 1.5 g/L fibrinogen (178) and a third sample comprises 0.75 g/L fibrinogen (180). The enlarged view of a relevant part of the data as displayed in FIG. 6B shows that at least in a particular range of frequencies as indicated by the boxed data points, the value ΔZ depends on the fibrinogen level of the analyzed sample 112. Said frequency range corresponds roughly to a range around 100 kHz. To facilitate interpretation, the horizontal axis values corresponding to 10 kHz (4.0) and 100 kHz (5.0) frequencies are marked with circles.

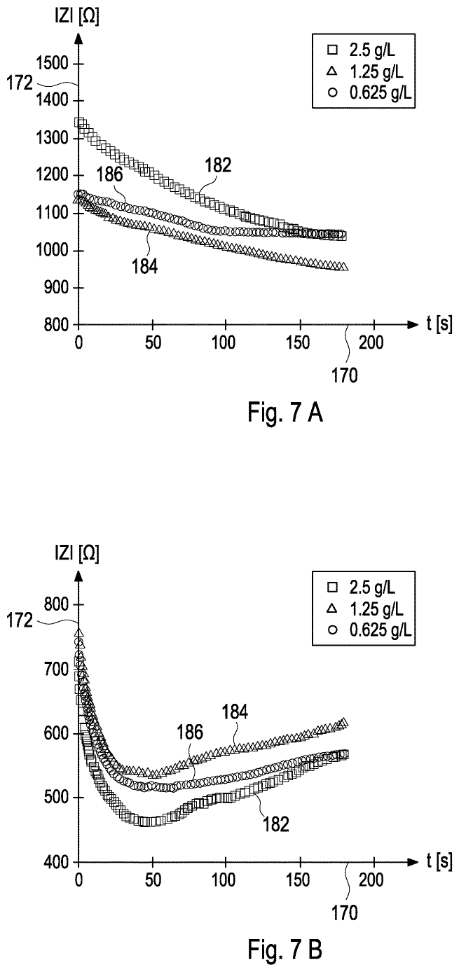

The data displayed in the measurement diagrams of FIGS. 3A, 3B, 4A, 4B, 5A, 5B, 6A and 6B are acquired using an electrode design, wherein each of the test electrodes 122 comprises two test electrode fingers 132 and each of the control electrodes 124 comprises two control electrode fingers 134. An example of such an electrode design is shown in FIG. 1A for a test element 110 comprising one pair of test electrodes 122 and one pair of control electrodes 124. The data displayed in the measurement diagrams of FIG. 7A, 7B, 8A, and 8B are acquired using an electrode design, wherein each of the test electrodes 122 comprises one test electrode finger 132 and each of the control electrodes 124 comprises one control electrode finger 134. An example of such an electrode design is shown in FIG. 1B for a test element 110 comprising one pair of test electrodes 122 and one pair of control electrodes 124.

FIG. 7A shows an electrical impedance control measurement, while FIG. 7B shows an electrical impedance test measurement. In both measurement diagrams, the time in seconds is plotted on the horizontal axis 170, while the magnitude of the impedance, |Z|, is plotted on the vertical axis 172 in ohm. Both measurement diagrams show data of an exemplary experiment with three samples 112 of different fibrinogen levels. The samples marked 182 comprise fibrinogen at a concentration of about 2.50 g/L. The samples marked 184 comprise fibrinogen at a concentration of about 1.25 g/L. The samples marked 186 comprise fibrinogen at a concentration of about 0.625 g/L. Both measurement diagrams show the course of ZI over a time of about 200 seconds at a voltage alternating at a frequency of 100 kHz. While the data displayed in FIG. 7A are acquired in the control region 120 in the absence of the detector compound 126, the data displayed in FIG. 7B are acquired in the test region 118 and thus in the presence of the detector compound 126. The three samples 182, 184, 186 in FIG. 3A, show a certain drift in the magnitude of their impedance |Z| over time. In FIG. 7B the three samples 182, 184, 186 first show a decrease in |Z|. After reaching a minimum at around 30 seconds, the magnitude of the impedance |Z| rises again, wherein the rise may reflect the clot-formation taking place when fibrinogen has been specifically cleaved by the detector compound. Further measurement data supporting this interpretation are shown in FIG. 10.

For the measurements shown in FIGS. 7A-7B, the detector compound 126 was present in the test region 118 from the beginning of the test and control measurements. The initial decrease in |Z| in the test measurement may reflect an induction period or activation period: Without being bound to theory, fibrinogen may be specifically cleaved into fibrin by the detector compound, specifically by a serine endopeptidase, more specifically by thrombin-like serine protease, most specifically by a batroxobin. The detector compound may specifically cleave the fibrinogen into fibrin-monomers resulting in a decrease of a viscosity of the sample and, thus, decreasing the impedance in the test region 118. After a certain amount of time, polymerization and/or cross-linking of the fibrin may enhance the sample's viscosity in the test region 118, thereby increasing the impedance. This behavior can be seen in FIG. 7B with the minimum in the impedance.

FIG. 8A shows a measurement diagram with the three shifted impedance test measurements. The measurement diagram in FIG. 8A is based on the measurement data displayed in FIGS. 7A-7B. In the measurement diagram of FIG. 8A, the time in seconds is plotted on the horizontal axis 170. On the vertical axis 172, ΔZ is plotted in ohm. ΔZ is determined by subtracting from the magnitude |Z| as determined in an impedance test measurement, the magnitude |Z| as determined in an impedance control measurement of the corresponding sample 112. Data of such corresponding samples 112 acquired at a voltage alternating at 100 kHz are displayed in FIGS. 7A-7B. As becomes obvious from FIG. 8A, a slope of the displayed curves varies corresponding to the initial fibrinogen level of the sample 112. Calculated straight lines 188, also referred to as fit curves, are fitted to the measurement data. The fit curves 188 are indicated as dotted lines in FIG. 8A. For the determination of the fit curve 188, measurement data starting with the minimum of the measurement curve is taken into account. The slope of the fit curve 188 corresponding to the sample of 2.50 g/L fibrinogen (182) is determined as 2.16, the slope of the fit curve 188 corresponding to the sample of 1.25 g/L fibrinogen (184) is determined as 1.40 and the slope of the fit curve corresponding to the sample of 0.625 g/L fibrinogen (186) is determined as 0.85.

In FIG. 8B, the values of the slopes as determined in FIG. 8A are plotted on the vertical axis against the fibrinogen concentrations of the corresponding samples 112 on the horizontal axis 170. FIG. 8B further displays a straight line as a fit curve 188 fitted to the three data points. The straight line equation (1) of the fit curve 188 in FIG. 8B is

y=0.6784*x+0.4764 (1)

The quality of the fit may be evaluated from the R² value, which is 0.9895. The fit curve 188 in FIG. 8B may allow to derive the fibrinogen level of a sample 112 from the measurement values of the sample 112 as e.g. acquirable with the impedance test system 146.

The evaluation unit 156 may be configured for determining the fibrinogen level by using a predetermined relation between the measurement results of the electrical impedance test measurement and the electrical impedance control measurement and the fibrinogen level. The fit curve 188 as described above by equation (1) may serve as a simplified example of such a predetermined relation. The relation between the measurement results of the electrical impedance test measurement and the electrical impedance control measurement and the fibrinogen level may be determined in a calibration process that may specifically comprise the steps as described above for compounding the relation. The calibration process may comprise further steps, including steps not described here. The calibration process may further comprise an iteration and/or reiteration of steps.

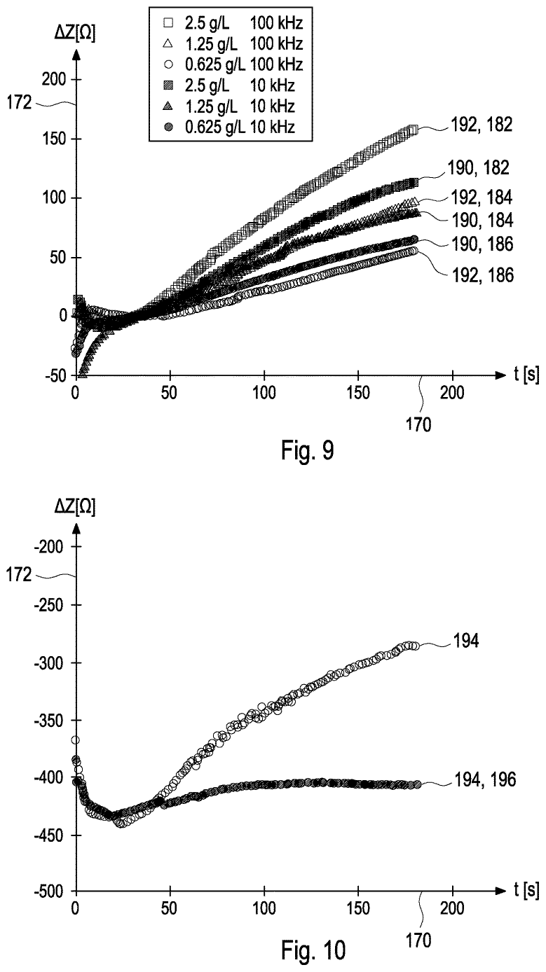

FIG. 9 shows a measurement diagram with a plurality of shifted impedance test measurements. In particular, FIG. 9 shows a measurement diagram with the time in seconds plotted on the horizontal axis 170. On the vertical axis 172, ΔZ is plotted in ohm. ΔZ is determined by subtracting from the magnitude |Z| as measured in the test region 118, the magnitude |ZI of the corresponding sample 112 in the control region 120. Each measurement curve was subsequently offset in y-direction such that the minimum is equal to zero. Samples 112 of three different concentrations, namely 2.50 g/L (marked 182), 1.25 g/L (marked 184) and 0.625 g/L (marked 186) were analyzed at two different alternating voltages, namely at 10 kHz (marked 190) and at 100 kHz (marked 192). In this analysis, the test electrodes 122 comprise two test electrode fingers 132 each, while the control electrodes 124 comprise two control electrode fingers 134 each. As becomes obvious from FIG. 9, AZ, and with ΔZ also the slope of the measurement curve, varies depending on the fibrinogen concentration of the sample 112 and the frequency of the alternating voltage applied. The values of the slopes of the samples 112 analyzed at 10 kHz are given in the following table:

TABLE 1
Slopes of the curves in FIG. 9 and corresponding
fibrinogen levels at 10 kHz.

| fibrinogen concentration | slope |
|---|---|
| 2.50 g/L | 0.550 |
| 1.25 g/L | 0.410 |
| 0.625 g/L | 0.309 |

The values of the slopes of the samples 112 analyzed at 100 kHz are given in the following table:

TABLE 2
Slopes of the curves in FIG. 9 and corresponding
fibrinogen levels at 100 kHz.

| fibrinogen concentration | slope |
|---|---|
| 2.50 g/L | 0.732 |
| 1.25 g/L | 0.461 |
| 0.625 g/L | 0.273 |

At both frequencies of the alternating voltage, the three different fibrinogen concentrations are resolved. At a voltage alternating at 100 kHz the differences between the normalized magnitudes of the impedance, AZ, and also the differences between the calculated slopes as given in the tables above are greater for samples 112 with different fibrinogen concentrations. Thus, the resolution for different fibrinogen concentrations is better at 100 kHz than at 10 kHz.

FIG. 10 shows a measurement diagram of an electrical impedance test measurement in the presence and in the absence of a fibrin polymerization blocker. In particular, FIG. 10 shows a measurement diagram with the time in seconds plotted on the horizontal axis 170. On the vertical axis 172, ΔZ is plotted in ohm. ΔZ is determined by subtracting from the magnitude |Z| as measured using a sample 112 in the test region 118 the magnitude |Z| of the corresponding sample 112 in the control region 120. The fibrinogen concentration of the samples 112 analyzed in this measurement is 3.1 g/L. Samples at this concentration are marked with the reference number 194. In the experiment shown, one of the samples 112 is analyzed in a test region 118 comprising in addition to the detector compound 126 a fibrin polymerization blocker, Pefabloc® FG, by LOXO GmbH. Pefabloc® FG binds with a high affinity to fibrinogen, inhibits fibrin polymerization, modifies the mechanical properties of fibrin clots and can dissociate non-stabilized fibrin gels. The samples marked 196 are analyzed in a test region 118 comprising Pefabloc® FG in addition to the detector compound 126. As can be seen in FIG. 10, the increase of ΔZ is largely suppressed in the presence of Pefabloc® FG, indicating that said increase is due to the property of the detector compound 126 of specifically cleaving fibrinogen.

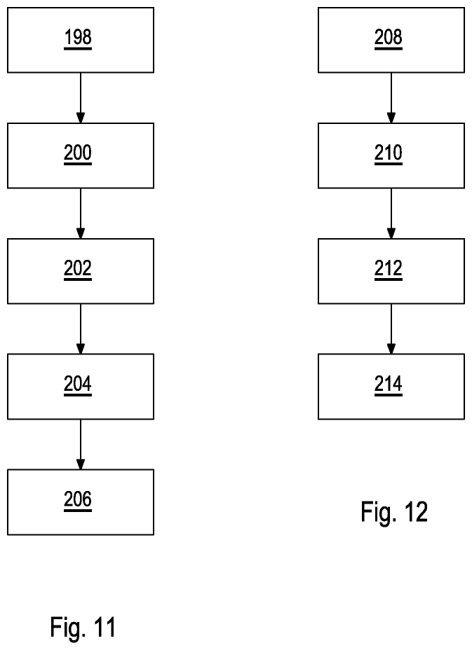

FIG. 11 shows a flow chart of an exemplary embodiment of an impedance method of ex-vivo detecting a fibrinogen level in a sample 112 of a bodily fluid. The method comprises the following steps, which, as an example, may be performed in the given order. A different order may also be possible. Further, one or more of the method steps may be performed once or repeatedly. Further, it may be possible to perform two or more of the method steps simultaneously or in a timely overlapping fashion. The method may comprise further method steps, which are not listed. The method steps, as shown in the flowchart in FIG. 11, are the following:
i. (Reference number 198) providing at least one test element 110 as described herein, such as in any of the embodiments described above and/or as in any one of the embodiments described in further detail below;
ii. (Reference number 200) applying the sample 112 of the bodily fluid to at least one application opening 130 of the capillary 116;
iii. (Reference number 202) performing at least one electrical impedance test measurement by using the test electrodes 122;
iv. (Reference number 204) performing at least one electrical impedance control measurement using the control electrodes 124; and
v. (Reference number 206) determining the fibrinogen level by using measurement results from the electrical impedance test measurement and from the electrical impedance control measurement.

The impedance method of ex-vivo detecting a fibrinogen level in a sample 112 of a bodily fluid may in particular be performed using the impedance test system 146 according to the present disclosure as described herein, such as in any of the embodiments described above and/or as in any one of the embodiments described in further detail below.

FIG. 12 shows a flow chart of an exemplary embodiment of a method of manufacturing a test element 110 for detecting a fibrinogen level in a sample 112 of a bodily fluid. The method comprises the following steps, which, as an example, may be performed in the given order. A different order may also be possible. Further, one or more of the method steps may be performed once or repeatedly. Further, it may be possible to perform two or more of the method steps simultaneously or in a timely overlapping fashion. The method may comprise further method steps, which are not listed. The method steps, as shown in the flowchart in FIG. 12, are:
I) (Reference number 208) providing at least one substrate 114 having disposed thereon at least two test electrodes 122 and at least two control electrodes 124;
II) (Reference number 210) disposing at least one detector compound 126 being capable of specifically cleaving fibrinogen;
III) (Reference number 212) providing at least one spacer layer 142;
IV) (Reference number 214) providing at least one cover layer 144; and
V) forming a sandwich setup 138 comprising, in the given order, the substrate 114, the spacer layer 142 and the cover layer 144, wherein a capillary 116 is provided in the sandwich setup 138 having sidewalls 164 formed by the spacer layer 142, with the test electrodes 122 and the control electrodes 124 facing the capillary 116, wherein at least one test region 118 and at least one control region 120 are formed within the capillary 116, wherein the test electrodes 122 are disposed within the test region 118, wherein the control electrodes 124 are disposed within the control region 120, and wherein the detector compound 126 is disposed within the test region 118.

In step II), said detector compound 126 may particularly be deposited by using a solution of the detector compound 126. Thus, the detector compound 126 may upon application be dissolved in a liquid or paste-like solution. The solution may, however, as part of the manufacturing process dry, harden or solidify such that the detector compound 126 may in the finished test element 110 be present in the test region 118 as a dried film or as part of the hardened detector compound layer 128. The detector compound 126 may in particular be a serine endopeptidase (E.C.3.4.21).

The test element 110 manufactured with the method of manufacturing a test element 110 for detecting a fibrinogen level in a sample 112 of a bodily fluid may specifically be a test element 110 according to the present disclosure.

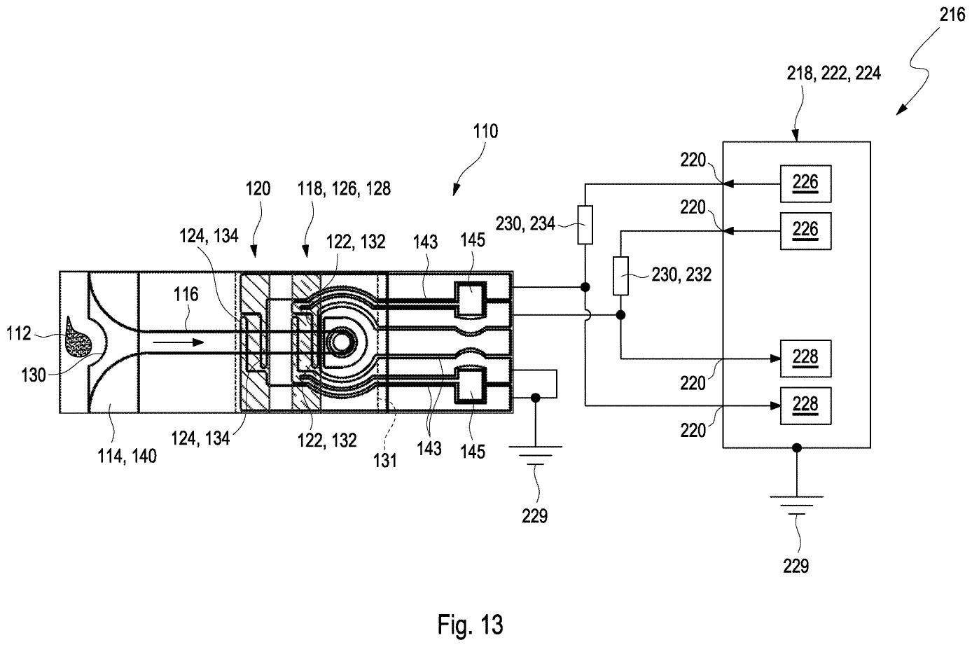

FIG. 13 shows an exemplary embodiment of a resistance test system 216 for detecting a fibrinogen level in a sample of a bodily fluid 112 in a schematic view. The resistance 216 comprises at least one test element 110 according to the present disclosure, such as according to any one of the embodiments shown in FIGS. 1A-1B and/or according to any other embodiment disclosed herein. Thus, for a detailed description of the test element 110 of the resistance test system 216, reference is made to the description of FIGS. 1A-1B.

The resistance test system 216 further comprises at least one test device 218 having contacts 220 for electrically contacting the test element 110. The test device 218 further has at least one voltage measurement unit 222. The voltage measurement unit 222 is configured for applying at least one voltage signal to the test electrodes 122 and the control electrodes 124 of the test element 110. The voltage measurement unit 222 is further configured for determining at least one test response signal of the test region 118. The voltage measurement unit 222 is further configured for determining at least one control response signal of the control region 120. For example, the test device 218 may comprise at least one microcontroller 224, specifically a mixed signal microcontroller, having digital outputs 226 to the test region 118 and the control region 120 and analog inputs 228 for receiving the response signals of the test region 118 and the control region 120, respectively. In the embodiment shown in FIG. 13, the test device 218 and/or the test element 110, specifically the electrodes in the control region 120 and in the test region 118, may be connected to ground 229.

As shown in FIG. 13, the resistance test system 216 may further comprise at least one reference resistor 230. Specifically, in this exemplary embodiment, the resistance test system 216 may comprise two reference resistors 230. The reference resistors 130 may be connected to the digital outputs 226 of the microcontroller 224. Thus, in this example, the test device 218 having two output contacts may be connected to the test region 118, specifically to the test electrodes 122, via a first reference resistor 232 and to the control region 120, specifically to the control electrodes 124, via a second reference resistor 234. Further, in the exemplary embodiment of FIG. 13, the resistance test system 216 may comprise at least one voltage divider. The voltage divider may comprise the reference resistor 230 and one of the test region 118 or the control region 120 of the test element 110. Specifically, as can be seen in FIG. 13, each reference resistor 230 may form with at least one of the test region 118 and the control region 120 one voltage divider.

The resistance test system 216 may further have at least one evaluation unit 236. The evaluation unit 236 may be or may comprise at least one evaluation unit 156 as defined above with respect to the impedance test system 146. The evaluation unit 236 may be configured for determining the fibrinogen level by using the test response signal and the control response signal. Specifically, the evaluation unit 236 may be configured, such as by software programming of at least one processor, for performing one or more evaluation operations for determining the fibrinogen level by using the test response signal and the control response signal. For example, the evaluation unit 236 may be configured for performing at least steps iv. and v. of a resistance method of ex-vivo detecting, specifically in-vitro detecting, a fibrinogen level in a sample of a bodily fluid 112, as will be outlined in further detail below.

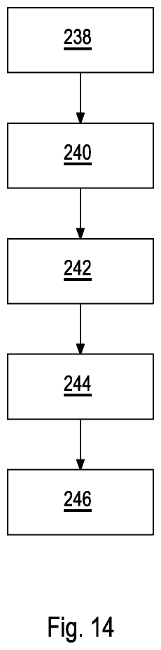

FIG. 14 shows a flow chart of an exemplary embodiment of a resistance method of ex-vivo detecting a fibrinogen level in a sample of a bodily fluid 112. The method comprises the following steps, which, specifically, may be performed in the given order. It shall be noted, however, that a different order may also be possible. Further, one or more of the method steps may be performed once or repeatedly. Further, it may be possible to perform two or more of the method steps simultaneously or in a timely overlapping fashion. The method may comprise further method steps, which are not listed. In some aspects, the method comprises the following steps:
i) (Denoted by reference number 238) providing at least one test element 110 according to the present disclosure, such as according to any one of the embodiments shown in FIGS. 1A-1B and/or according to any other embodiment disclosed herein;
ii) (Denoted by reference number 240) applying the sample of the bodily fluid 112 to at least one application opening 130 of the capillary 116;
iii) (Denoted by reference number 242) applying at least one voltage signal to the test electrodes 122 and the control electrodes 124 of the test element 110;
iv) (Denoted by reference number 244) determining at least one test response signal of the test region 118 and at least one control response signal of the control region 120; and
v) (Denoted by reference number 246) determining the fibrinogen level by using the test response signal and the control response signal.

Specifically, in the method, the resistance test system 216 according to the present disclosure, such as according to the embodiment shown in FIG. 13 and/or according to any one of the embodiments disclosed in further detail below, may be used. Thus, for a description of the resistance test system 216 to be used in the resistance method, reference is made to the description of FIG. 13.

Results of the resistance method are shown in FIGS. 15A-18. Thus, in the following FIG. 15A-18 are described in conjunction with FIG. 14.

FIG. 15A-15F show equivalent circuits of the test region 118 and the control region 120 and respective diagrams of voltage signals and response signals. Specifically, FIG. 15A shows an equivalent circuit of the control region 120 with the voltage signal applied to the control region 120 denoted as U_{P_CZ}, the resistance of the reference resistor 230, 234 denoted as R_{ref} and the impedance of the control region 120 denoted as Z_{CZ}. FIG. 15B shows the applied voltage signal (solid line in FIG. 15B) and the control response signal of the control region 120 (dashed line in FIG. 15B) as a function of time. FIG. 15C shows a magnification on the rising flank of the control response signal of control region 120 shown in FIG. 15B.

As shown in FIG. 15B, the voltage signal may comprise a rectangular voltage pulse applied to the pair of control electrodes 124 in the control region 120, the voltage pulse having a pulse duration in the range of 1 μs to 500 μs, more specifically of 200 μs. The control region 120 and the reference resistor 230 may be arranged to form a voltage divider, as outlined above. The resistance R_{ref} of the reference resistor 23, 234 may be known or predetermined. The impedance Z_{CZ} may be formed by the sample of the bodily fluid 112 and the control electrodes 124 in the control region 120. The potential U_{CZ} over Z_{CZ} (denoted by reference number 248) may be measured. Specifically, the potential U_{CZ} may be the control response signal of the control region 120 determined in step iv. of the resistance method. Since it is assumed that no coagulation is present in the control region 120, the capacitive behavior of the control response signal (denoted by reference number 250) may arise from the hematocrit and/or other dielectric properties of the sample of the bodily fluid 112, which are responsible for a double layer capacitance in the control response signal.

Further, FIG. 15D shows an equivalent circuit of the test region 118 with the voltage signal applied to the test region 118 denoted as U_{P_RZ}, the resistance of the reference resistor 230, 232 denoted as R_{Ref} and the impedance of the test region 118 denoted as Z_{RZ}. FIG. 15E shows the applied voltage signal (solid line in FIG. 15E) and the test response signal of the test region 118 (dashed line in FIG. 15E) as a function of time. FIG. 15F shows a magnification on the rising flank of the test response signal of the test region 118 shown in FIG. 15E. As can be seen in FIG. 15E, the voltage signal applied to the test region 118, specifically to the test electrodes 122 of the test region 118, may comprise an identical rectangular voltage pulse being applied to the control region 120. The two identical voltage pulses may be applied sequentially with a delay of about 1 s at the control region 120 and the test region 118. The pulse amplitude U_{P_CZ} of the control region 120 may have the same magnitude and form as the pulse amplitude U_{P_RZ} of the test region 118.

Further, as shown in FIG. 15D, the test region 118 and the reference resistor 230, 232 may be arranged to form a voltage divider, as outlined above. The resistance R_{ref} of the reference resistor 230, 232 may be known or predetermined. The impedance Z_{RZ} of the test region 118 may be formed by the sample of the bodily fluid 112 and the test electrodes 122 in the test region 118. The potential U_{RZ} over Z_{RZ} (denoted by reference number 252) may be measured. Specifically, the potential U_{RZ} may be the test response signal of the test region 118 determined in step iv. of the resistance method. The detector compound 126 may be present in the test region 118 and, thus, coagulation may occur and a change of the dielectric properties may be high in comparison to the changes of the control region 120. Thus, a double layer capacitance of the test region 118 may be much higher than in the control region 120 indicated by a much higher capacitive part at the rising flank of the voltage pulse (denoted by reference number 254).

In the control region 120, the current i_{CZ} flowing through the reference resistor 230, 234 and the sample of the bodily fluid 112 may be determined using the following equation, wherein the applied voltage signal U_{P_CZ} in the control region 120 and the resistance R_{ref} of the reference resistor 230, 234 are known and the potential U_{CZ} in the control region 120 is measured:

i_{CZ}=(U_{P_CZ}−U_{CZ})/R_{ref}.

An equivalent series resistance (ESR) value of the control region 120 may comprise the real part of the impedance Z_{CZ} which can be calculated from the measured potential U_{CZ} and the current from i_{CZ} using Ohm's law:

ESR_{CZ}=Re(Z_{CZ})=R_{ref}·U_{CZ}/(U_{P_CZ}−U_{CZ}).

In the test region 118, the current i_{RZ} flowing through the reference resistor 230, 232, specifically having the same resistance as the reference resistor 230 234 in the control region 120, may be determined using the following equation, wherein the applied voltage signal U_{P_RZ} in the test region 118 and the resistance R_{ref} of the reference resistor are known and the potential U_{RZ} in the test region 118 is measured:

i_{RZ}=(U_{P_RZ}−U_RZ)/R_{ref}.

An ESR value of the test region 118 may be determined according to:

ESR_{RZ}=Re(Z_{RZ})=R_{ref}·U_{RZ}/(U_{P_RZ}−U_{RZ}).

Thus, both ESR values of the control region 120 and the test region 118 may depend directly on the measured potentials U_{CZ} and U_{RZ}. The point of interest being required to evaluate the ESR pulses may be the point in time at ti where the resistive flank of the response signal undergoes a flection and is getting capacitive. In order to find the point of interest, the control response signal U_{CZ}(t) may be subtracted from the test response signal U_{RZ}(t) to get a differential pulse dU with:

dU=U_{RZ}(t)−U_{CZ}(t).

The point of interest may be determined by determining a minimum in the difference of the test response signal and the control response signal, which may reflect the transition point where the response signals have reached an end of the resistive part of the impedance. This is exemplarily shown in FIG. 16. FIG. 16 shows a diagram of test response signals and control response signals as a function of time. Specifically, in the diagram of FIG. 16, a plurality of test response signals 256, a plurality of control response signals 258 and the difference 260 between the plurality of test response signals 256 and the plurality of control response signals 258 are shown as a function of time 262. As can be seen in FIG. 16, the beginning of the ESR flank may determine the transition of the resistive properties to the capacitive behavior of the complex impedance of the sample of the bodily fluid 112. The transition may be determined by the minimum in the difference 260 between the plurality of test response signals 256 and the plurality of control response signals 258, as indicated by the arrow in FIG. 16.

Turning back to FIG. 14, the voltage signal applied in step iii. of the resistance method may, as an example, comprise a sequence of voltage pulses. Each voltage pulse may be applied for a pulse duration of 1 ns to 1 ms, specifically of 1 μs to 500 μs, more specifically of 200 μs. Each voltage pulse may have a pulse amplitude in the range from 0.1 V to 10 V, specifically from 1 V to 3 V. The voltage pulses of the sequence of voltage pulses may specifically comprise equal pulse amplitude and/or pulse duration. In step iv., a plurality of test response signals 256 of the test region 118 and a plurality of control response signals 258 of the control region 120 may be determined for each voltage pulse of the sequence of voltage pulses. Step v. may comprise determining a minimum of the difference 260 between the plurality of test response signals 256 and the plurality of control response signals 258 for each voltage pulse, specifically determining the minimum in the difference as discussed with respect to FIG. 16. Further, the method may comprise averaging the minima of the difference 260 between the plurality of test response signals 256 and the plurality of control response signals 258 over the sequence of voltage pulses.

Further, steps iii) and iv) may be performed repeatedly during at least one measurement duration. This is shown in FIG. 17. Specifically, FIG. 17 shows a diagram of a plurality of averaged minima 264 of the difference between the plurality of test response signals 256 and the plurality of control response signals 258 as a function of time 262. In the example of FIG. 17, the measurement duration was 180 s.

Further, as an optional step, the method may start with heating up the test element 110 and, after reaching a predetermined setting point, e.g. after reaching 37.0 °C, determining a wetting of the control electrodes 124 and the test electrodes 122. In case the wetting of the control electrodes 124 and the test electrodes 122 by the sample of the bodily fluid 112 is determined, the method may proceed with steps iii) to v) of the resistance method, such as, in this example, with the applying of the sequence of voltage pulses. Specifically, the sequence of voltage pulses may be applied multiple times within the measurement duration. Steps iii) and iv) may specifically be performed in regular time intervals within the measurement duration. In the example of FIG. 17, the method may comprise applying a sequence of a number of 200 voltage pulses, each voltage pulse being applied for 200 μs, wherein the sequence of voltage pulses is applied within a time interval of 100 ms. During each single voltage pulse, the temperature of the test element 110, specifically the temperature of the test region 118 and/or the temperature of the control region 120, may be determined, controlled and/or regulated. Further, each single voltage pulse may be evaluated by calculating the differential pulse dU and the minimum dU for each single voltage pulse. This may be done 200 times within 100 ms. At the end of the 100 ms time interval, the mean of 200 measured temperatures und the mean of 200 differential pulses dU may be calculated and may be stored as the result of one measurement point. The result curve may comprise the plurality of averaged minima 264 of the difference between the plurality of test response signals 256 and the plurality of control response signals 258 as a function of time 262. Thus, the curve of FIG. 18 shows the averaged minima dU plotted vs. the measurement duration of 180 s.

The method may further comprise determining at least one slope of a linear regime 266 evolving from a minimum 268 of the plurality of averaged minima 264 of the difference between the plurality of test response signals 256 and the plurality of control response signals 258. The linear regime 266 may be limited by at least one predetermined range following the minimum 268 of the plurality of averaged minima 264 of the difference between the plurality of test response signals 256 and the plurality of control response signals 258. The predetermined range may comprise an absolute predetermined range or a relative predetermined range, specifically a predetermined range comprising a part of the averaged minima 264 following the minimum 268 of the plurality of averaged minima. In the example of FIG. 18, the predetermined range may comprise a number of 25% of the averaged minima 264 starting from the minimum 268 of the plurality of averaged minima to the end of the measurement duration. Thus, the evaluation of the curve may comprise determining the slope beginning at the minimum 268 of the curve to 25% of the rest of the curve.

Further, the fibrinogen level may be determined by using the slope of the linear regime 266. The effect of different fibrinogen levels on the slope of the linear regime can be seen in FIG. 18. FIG. 18 shows a diagram of a plurality of averaged minima 264 for different fibrinogen levels. The different levels of fibrinogen were obtained by diluting a blood sample under a constant hematocrit. The erythrocytes were separated from the plasma. Then, the plasma was diluted with NaCl in order to obtain different levels of fibrinogen. Subsequently, the erythrocytes were added to obtain the different levels of diluted plasma and the fibrinogen levels were measured by using the resistance method as described above. The results are shown in FIG. 18 for high fibrinogen level 270, for medium fibrinogen level 272, for low fibrinogen level 274 and for high fibrinogen level without the detector compound 276 in the test region 118.

Specifically, the fibrinogen level may be determined using at least one relation relating the slope of the linear regime to the fibrinogen level. The relation, as an example, may be or may comprise at least one analytical relation, such as a relation being programmed as an equation, wherein parameters of the equation are determined, e.g. by calibration measurements. Additionally or alternatively, the relation, as an example, may be programmed as a lookup list or lookup table, wherein fibrinogen levels are given for various measurement results, such as for various slopes of the linear regime. Various options are feasible and generally known to the skilled person.

The result curves of the plurality of averaged minima 264 shown in FIG. 18 had a slope of 0.06 for the high fibrinogen level 270, a slope of 0.03 for the medium fibrinogen level 272 and a slope of 0.004 for the low fibrinogen level 274 in the respective linear regime 266. The result curve for the high fibrinogen level without the detector compound 276 showed no response and, thus, no minimum in the plurality of averaged minima 264.

Additionally, as an optional step before step iii., the resistance method of FIG. 14 may comprise determining a wetting of at least one of the control electrodes 124 and the test electrodes 122. Steps iii. to v. may be performed at at least one predetermined point in time after determination of the wetting. The determining of the wetting may specifically be performed prior to step iii). FIGS. 19A and 19B show diagrams of determining the wetting of control electrodes 124 and test electrodes 122. In FIGS. 19A-19B, exemplary measurements for determining the wetting using the resistance test system 216 are shown, wherein the response signals of the test region 118 are denoted by reference number 278, the response signals of the control region 120 are denoted by reference number 280, and the difference dU between the response signals of the test region 118 and the response signals of control region 120 is denoted by reference number 282. In the diagrams of FIGS. 19A-19B, the counts 284 of these signals are shown as a function of time 262, wherein one count refers to an amplitude of 0.8 mV. The time difference between two data points was 1.08 μs.

After applying the sample of the bodily fluid 112 to the test element 110, the sample of the bodily fluid 112 may pass first the control region 120 where the determining of the wetting starts. The test device 218 may be configured for recognizing the presence of the sample of the bodily fluid 112 by applying a sequence of voltage pulses applied to the test region 118 and to the control region 120. In the dry state, when no sample of bodily fluid 112 is applied to the test element 110, the response signals 280 of the control region 120 may have a nearly rectangular shape since there is only air between the control electrodes 124 and, thus, the capacitance of the control region 120 is small compared to the wetted state. The capacitance may depend only on the coating of the electrodes. The response signals 278 of the test region 118 may also have a rectangular shape and may have the same amplitude compared to the response signals of the control region 120. A difference of the response signals 282 of the control region 120 and test region 118 may be determined and may give a straight line close to zero, as can be seen in FIG. 19A.

The situation after applying the sample of the bodily fluid 112 to the test element 110 is shown in FIG. 19B. After applying the sample of the bodily fluid 112, a difference in the capacitive charging may be visible. The test region 118 may show stronger capacitive properties than the control region 120. Thus, the capacitive properties of the test region 118 and the control region 120 may be different. The double layer capacitance of the test region 118 may be higher than of the control region 120 since the detector compound 126 may change the viscosity of the sample of the bodily fluid 112 in the test region 118. The difference of the response signals 282 of the control region 120 and test region 118 may show a negative peak, as can be seen in FIG. 19B, which may be used to detect wetting of the control electrodes 124 and the test electrodes 122.

Summarizing and without excluding further possible embodiments, the following embodiments may be envisaged:
Embodiment 1. A test element for detecting a fibrinogen level in a sample of a bodily fluid, the test element comprising at least one substrate and at least one capillary for receiving and transporting the sample, the test element further comprising at least one test region and at least one control region within the capillary, wherein the test element, in the test region, comprises at least two test electrodes, wherein the test element, in the control region, comprises at least two control electrodes, wherein the test element further comprises, in the test region, at least one detector compound, the detector compound being capable of specifically cleaving fibrinogen.
Embodiment 2. The test element according to the preceding embodiment, wherein the test element, in the control region, is devoid of the at least one detector compound.
Embodiment 3. The test element according to any one of the preceding embodiments, wherein the detector compound is a serine endopeptidase (E.C.3.4.21).
Embodiment 4. The test element according to the preceding embodiment, wherein said serine endopeptidase is a thrombin-like serine protease.
Embodiment 5. The test element according to the preceding embodiment, wherein said thrombin-like serine protease is having venombin A activity.
Embodiment 6. The test element according to the preceding embodiment, wherein said thrombin-like serine protease is a batroxobin.
Embodiment 7. The test element according to any one of the preceding embodiments, wherein the detector compound is disposed on the test electrodes.
Embodiment 8. The test element according to any one of the preceding embodiments, wherein the detector compound is comprised in at least one detector compound layer.
Embodiment 9. The test element, the detector compound layer being disposed on at least one side wall of the capillary, extending over the entire length of the test region in a direction of longitudinal extension of the capillary.
Embodiment 10. The test element according to any one of the preceding embodiments, the test region and the control region being disposed in the test element in accordance with a setup selected from the group consisting of:
   the test region and the control region are disposed in the same capillary, wherein the control electrodes and the test electrodes are disposed within the capillary such that the sample of bodily fluid entering the capillary passes the control electrodes before passing the test electrodes; and
   the test element comprises at least two capillaries, wherein the test region and the control region are disposed in different capillaries, specifically in different capillaries having a common application opening.
Embodiment 11. The test element according to the preceding embodiment, wherein the test region and the control region are disposed in the same capillary, wherein the control electrodes and the test electrodes are spaced apart by a minimum distance of 1.0 mm to 2.5 mm, specifically by a minimum distance of 1.6 mm to 2.0 mm.
Embodiment 12. The test element according to embodiment 10, wherein the test element comprises at least two capillaries, with the test region and the control region being disposed in different capillaries, wherein a minimum distance between the at least one application opening of the capillaries and the control electrodes and a minimum distance between the at least one application opening and the test electrodes is essentially identical.
Embodiment 13. The test element according to any one of the three preceding embodiments, wherein the test element comprises at least two capillaries, with the test region and the control region being disposed in different capillaries, wherein the capillaries comprise a common application opening.
Embodiment 14. The test element according to any one of the preceding embodiments, wherein the test element is a strip-shaped test element, wherein the substrate is a flexible substrate.
Embodiment 15. The test element according to any one of the preceding embodiments, wherein the test element further comprises at least one cover layer and at least one spacer layer disposed between the substrate and the cover layer, wherein the spacer layer forms sidewalls of the capillary.
Embodiment 16. The test element according to any one of the preceding embodiments, wherein the test element contains at least one application opening of the capillary and further contains at least one vent opening of the capillary, wherein the test region and the control region are disposed in between the application opening and the vent opening.
Embodiment 17. The test element according to any one of the preceding embodiments, wherein the test electrodes and the control electrodes are disposed on the substrate, specifically on the same side of the same substrate.
Embodiment 18. The test element according to any one of the preceding embodiments, wherein the test region and the control region have essentially identical dimensions.
Embodiment 19. The test element according to any one of the preceding embodiments, wherein the test electrodes and the control electrodes have an essentially identical geometry.
Embodiment 20. The test element according to any one of the preceding embodiments, wherein the test electrodes contain test electrode fingers spaced apart in a direction of longitudinal extension of the capillary.
Embodiment 21. The test element according to the preceding embodiment, wherein the test electrode fingers are spaced apart by 0.10 mm to 1.00 mm, specifically by 0.20 mm to 0.80 mm.
Embodiment 22. The test element according to any one of the two preceding embodiments, wherein the test electrode fingers have a width of 0.1 mm to 0.5 mm, specifically of 0.25 mm to 0.4 mm.
Embodiment 23. The test element according to any one of the three preceding embodiments, wherein the test electrodes comprise two test electrodes, the two test electrodes having interleaving test electrode fingers.
Embodiment 24. The test element according to any one of the preceding embodiments, wherein the control electrodes contain control electrode fingers spaced apart in a direction of longitudinal extension of the capillary.
Embodiment 25. The test element according to the preceding embodiment, wherein the control electrode fingers are spaced apart by 0.10 mm to 1.00 mm, specifically by 0.20 mm to 0.80 mm.
Embodiment 26. The test element according to any one of the two preceding embodiments, wherein the control electrode fingers have a width of 0.1 mm to 0.5 mm, specifically of 0.25 mm to 0.4 mm.
Embodiment 27. The test element according to any one of the three preceding embodiments, wherein the control electrodes comprise two control electrodes, the two control electrodes having interleaving control electrode fingers.
Embodiment 28. The test element according to the preceding embodiment, wherein the test electrodes and the control electrodes comprise contact leads having contact pads.
Embodiment 29. The test element according to the preceding embodiment, wherein the contact pads of the test electrodes and the control electrodes are disposed on the same side of the substrate.
Embodiment 30. The test element according to any one of the two preceding embodiments, wherein at least a first one of the test electrodes and at least a first one of the control electrodes are electrically contacted via a common contact lead, wherein at least a second one of the test electrodes and at least a second one of the control electrodes are electrically contacted via separate contact leads.
Embodiment 31. The test element according to any one of the preceding embodiments, wherein the capillary has a width of 0.5 mm to 2.5 mm, specifically of 1.0 to 2.0 mm, more specifically of 1.5 mm.
Embodiment 32. The test element according to any one of the preceding embodiments, wherein the capillary has a height of 50 μm to 500 μm, specifically of 100 μm to 300 μm, more specifically of 175 μm.
Embodiment 33. An impedance test system for detecting a fibrinogen level in a sample of a bodily fluid, the impedance test system comprising:
   at least one test element according to any one of the preceding embodiments; and
   at least one test device having contacts for electrically contacting the test element, the test device further having at least one impedance measurement unit, wherein the impedance measurement unit is configured for performing at least one electrical impedance test measurement using the test electrodes, wherein the impedance measurement unit is further configured for performing at least one electrical impedance control measurement using the control electrodes.
Embodiment 34. The impedance test system according to the preceding embodiment, the impedance test system further having at least one evaluation unit, the evaluation unit being configured for determining the fibrinogen level by using measurement results from the electrical impedance test measurement and from the electrical impedance control measurement.
Embodiment 35. The impedance test system according to the preceding embodiment, wherein the evaluation unit is configured for determining the fibrinogen level by using the result of the electrical impedance control measurement as a basal impedance for taking into account at least one specific sample property of the bodily fluid other than the fibrinogen level, specifically a hematocrit value.
Embodiment 36. The impedance test system according to any one of the two preceding embodiments, wherein the evaluation unit is configured for determining the fibrinogen level by using a predetermined relation between the measurement results of the electrical impedance test measurement and the electrical impedance control measurement and the fibrinogen level.
Embodiment 37. The impedance test system according to any one of the preceding embodiments referring to an impedance test system, the impedance test system further being configured for determining a wetting of at least one of the control electrodes and the test electrodes, the impedance test system further being configured for performing the electrical impedance test measurement and the electrical impedance control measurement at at least one predetermined point in time after the wetting.
Embodiment 38. An impedance method of ex-vivo detecting, specifically in-vitro detecting, a fibrinogen level in a sample of a bodily fluid, comprising:
   providing at least one test element according to any one of the preceding embodiments referring to a test element;
   applying the sample of the bodily fluid to at least one application opening of the capillary;
   performing at least one electrical impedance test measurement by using the test electrodes;
   performing at least one electrical impedance control measurement using the control electrodes; and
   determining the fibrinogen level by using measurement results from the electrical impedance test measurement and from the electrical impedance control measurement.
Embodiment 39. The impedance method according to the preceding embodiment, wherein the impedance test system according to any one of the preceding embodiments referring to an impedance test system is used.
Embodiment 40. A resistance test system for detecting a fibrinogen level in a sample of a bodily fluid, the test system comprising:
   at least one test element according to any one of the preceding embodiments referring to a test element; and
   at least one test device having contacts for electrically contacting the test element, the test device further having at least one voltage measurement unit, wherein the voltage measurement unit is configured for applying at least one voltage signal to the test electrodes and the control electrodes of the test element, wherein the voltage measurement unit is further configured for determining at least one test response signal of the test region, wherein the voltage measurement unit is further configured for determining at least one control response signal of the control region.
Embodiment 41. The resistance test system according to the preceding embodiment, the resistance test system further having at least one evaluation unit, the evaluation unit being configured for determining the fibrinogen level by using the test response signal and the control response signal.
Embodiment 42. The resistance test system according to any one of the two preceding embodiments, wherein the resistance test system further comprises at least one reference resistor, wherein the test device is configured for electrically contacting the test element via the reference resistor.
Embodiment 43. The resistance test system according to the preceding embodiment, wherein the resistance test system further comprises at least one voltage divider, the voltage divider comprising the reference resistor and one of the test region or the control region of the test element.
Embodiment 44. The resistance test system according to any one of the four preceding embodiments, wherein the voltage measurement unit is configured for separately controlling the at least one voltage signal applied to the test electrodes and the control electrodes of the test element.
Embodiment 45. The resistance test system according to any one of the five preceding embodiments, wherein the test device comprises at least one microcontroller.
Embodiment 46. The resistance test system according to any one of the six preceding embodiments, wherein the resistance test system is further configured for determining a wetting of at least one of the control electrodes and the test electrodes, the resistance test system further being configured for applying the at least one voltage signal to the test electrodes and the control electrodes of the test element at at least one predetermined point in time after the wetting.
Embodiment 47. A resistance method of ex-vivo detecting, specifically in-vitro detecting, a fibrinogen level in a sample of a bodily fluid, comprising
   providing at least one test element according to any one of the preceding embodiments referring to a test element;
   applying the sample of the bodily fluid to at least one application opening of the capillary;
   applying at least one voltage signal to the test electrodes and the control electrodes of the test element;
   determining at least one test response signal of the test region and at least one control response signal of the control region; and
   determining the fibrinogen level by using the test response signal and the control response signal.
Embodiment 48. The resistance method according to the preceding embodiment, wherein the resistance test system according to any one of the preceding embodiments referring to a resistance test system is used.
Embodiment 49. The resistance method according to any one of the two preceding embodiments, wherein the voltage signal comprises at least one of: a square wave signal; a voltage pulse; a sequence of voltage pulses.
Embodiment 50. The resistance method according to any one of the three preceding embodiments, wherein the voltage signal comprises a sequence of voltage pulses, wherein, in step iv., a plurality of test response signals of the test region and a plurality of control response signals of the control region is determined for each voltage pulse of the sequence of voltage pulses.
Embodiment 51. The resistance method according to the preceding embodiment, wherein step v. comprises determining a minimum of a difference between the plurality of test response signals and the plurality of control response signals for each voltage pulse.
Embodiment 52. The resistance method according to the preceding embodiment, further comprising averaging the minima of the difference between the plurality of test response signals and the plurality of control response signals over the sequence of voltage pulses.
Embodiment 53. The method according to any one of the three preceding embodiments, wherein steps iii) and iv) are performed repeatedly during at least one measurement duration, specifically in regular time intervals within the measurement duration.
Embodiment 54. The method according to the preceding embodiment, further comprising determining a plurality of averaged minima of the difference between the plurality of test response signals and the plurality of control response signals within the measurement duration as a function of time.
Embodiment 55. The method according to the preceding embodiment, further comprising determining at least one slope of a linear regime evolving from a minimum of the plurality of averaged minima of the difference between the plurality of test response signals and the plurality of control response signals.
Embodiment 56. The method according to the preceding embodiment, wherein the linear regime is limited by at least one predetermined range following the minimum of the plurality of averaged minima of the difference between the plurality of test response signals and the plurality of control response signals.
Embodiment 57. The method according to the preceding embodiment, wherein the predetermined range comprises an absolute predetermined range or a relative predetermined range, specifically a predetermined range comprising a part of the averaged minima following the minimum of the plurality of averaged minima.
Embodiment 58. The method according to any one of the three preceding embodiments, wherein the fibrinogen level is determined by using the slope of the linear regime.
Embodiment 59. The resistance method according to any one of the eleven preceding embodiments, further comprising determining a wetting of at least one of the control electrodes and the test electrodes, wherein steps iii) to v) are performed at at least one predetermined point in time after determination of the wetting.
Embodiment 60. The method according to any one of the twelve preceding embodiments, further comprising controlling at least one temperature of at least one of the test region and the control region of the test element.
Embodiment 61. A computer program comprising instructions which, when the program is executed by a resistance test system according to any one of the preceding embodiments referring to a resistance test system, cause the resistance test system to perform the resistance method of ex-vivo detecting a fibrinogen level in a sample of a bodily fluid according to any one of the preceding embodiments referring to a resistance method.
Embodiment 62. A computer-readable storage medium, specifically a non-transient computer readable storage medium, comprising instructions which, when the instructions are executed by a resistance test system according to any one of the preceding embodiments referring to a resistance test system, cause the resistance test system to perform the resistance method of ex-vivo detecting a fibrinogen level in a sample of a bodily fluid according to any one of the preceding embodiments referring to a resistance method.
Embodiment 63. A method of manufacturing a test element for detecting a fibrinogen level in a sample of a bodily fluid, the method comprising:
   providing at least one substrate having disposed thereon at least two test electrodes and at least two control electrodes;
   disposing at least one detector compound being capable of specifically cleaving fibrinogen and optionally being capable of activating fibrinogen;
   providing at least one spacer layer;
   providing at least one cover layer; and
   forming a sandwich setup comprising, in the given order, the substrate, the spacer layer and the cover layer, wherein a capillary is provided in the sandwich setup having sidewalls formed by the spacer layer, with the test electrodes and the control electrodes facing the capillary, wherein at least one test region and at least one control region are formed within the capillary, wherein the test electrodes are disposed within the test region, wherein the control electrodes are disposed within the control region, and wherein the detector compound is disposed within the test region.
Embodiment 64. The method according to the preceding embodiment, wherein, in step II), said detector compound is deposited by using a solution of the detector compound.
Embodiment 65. The method according to the preceding embodiment, wherein the detector compound is a serine endopeptidase (E.C.3.4.21).
Embodiment 66. The method according to any one of the three preceding embodiments, wherein the test element is a test element according to any one of the preceding embodiments referring to a test element.

The content of any patent, patent application, and/or publication cited in this specification is incorporated by reference in its entirety, with the exception of lexicographical definitions used therein. Any lexicographical definition in a cited patent, patent application, or publication that is not also expressly repeated in the instant application should not be treated as such and should not be read as defining any terms appearing in the accompanying claims. Any terms not specifically defined within the instant application, including terms of art, are interpreted as would be understood by one of ordinary skill in the relevant art; thus, is not intended for any such terms to be defined by a lexicographical definition in any cited art, whether or not incorporated by reference herein, including but not limited to, published patents and patent applications. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

### LIST OF REFERENCE NUMBERS

110 test element
112 sample of bodily fluid
114 substrate
116 capillary
118 test region
120 control region
122 test electrode
124 control electrode
126 detector compound
128 detector compound layer
130 application opening
131 heating zone
132 test electrode finger
133 heating element
134 control electrode fingers
135 test element receptacle
136 electrode foil
138 layer composition
140 layer
142 spacer layer
143 contact lead
144 cover layer
145 contact pad
146 impedance test system
148 test device
150 contacts
152 impedance measurement unit
153 current source
154 potentiostat
155 voltage source
156 evaluation unit
157 current measurement device
158 processor
159 voltage measurement device
160 fibrin clot
162 support surface
164 sidewall
166 first portion
168 second portion
170 horizontal axis
172 vertical axis
174 addition of detector compound
176 fibrinogen concentration of 3 g/L
178 fibrinogen concentration of 1.5 g/L
180 fibrinogen concentration of 0.75 g/L
182 fibrinogen concentration of 2.50 g/L
184 fibrinogen concentration of 1.25 g/L
186 fibrinogen concentration of 0.625 g/L
188 fit curve
190 10 kHz
192 100 kHz
194 fibrinogen concentration of 3.1 g/L
196 Pefabloc® FG
198 step i)
200 step ii)
202 step iii)
204 step iv)
206 step v)
208 step I.
210 step II.
212 step III.
214 step IV.
216 resistance test system
218 test device
220 contacts
222 voltage measurement unit
224 microcontroller
226 digital output
228 analog input
229 ground
230 reference resistor
232 first reference resistor
234 second reference resistor
236 evaluation unit
238 providing a test element
240 applying the sample of the bodily fluid
242 applying a voltage signal
244 determining a test response signal and a control response signal
246 determining the fibrinogen level
248 resistive part of the control response signal
250 capacitive part of the control response signal
252 resistive part of the test response signal
254 capacitive part of the test response signal
256 plurality of test response signals
258 plurality of control response signals
260 difference between test response signals and control response signals
262 time
264 averaged minima of the difference between test response signals and control response signals
266 linear regime
268 minimum of the plurality of averaged minima
270 high fibrinogen level
272 medium fibrinogen level
274 low fibrinogen level
276 high fibrinogen level without the detector compound
278 response signals of the test region
280 response signals of the control region
282 difference between the response signals of the test region and the response signals of control region
284 counts

1. A test element (110) for detecting a fibrinogen level in a sample (112) of a bodily fluid, the test element (110) comprising:
at least one substrate (114) and at least one capillary (116) for receiving and transporting the sample (112),
at least one test region (118) and at least one control region (120) within the capillary (116), wherein the test element (110), in the test region (118), comprises at least two test electrodes (122), wherein the test element (110), in the control region (120), comprises at least two control electrodes (124),
wherein the test element (110) further comprises, in the test region (118), at least one detector compound (126), the detector compound (126) being capable of specifically cleaving fibrinogen,
wherein the test region (118) and the control region (120) are disposed in the test element (110) in accordance with a setup where the test region (118) and the control region (120) are disposed in the same capillary (116), wherein the control electrodes (124) and the test electrodes (122) are disposed within the capillary (116) such that the sample (112) of bodily fluid entering the capillary (116) passes the control electrodes (124) before passing the test electrodes (122).

2. The test element (110) according to claim 1, wherein the detector compound (126) is a serine endopeptidase (E.C.3.4.21).

3. The test element (110) according to claim 2, wherein said serine endopeptidase is a thrombin-like serine protease.

4. The test element (110) according to claim 3, wherein said thrombin-like serine protease is having venombin A activity.

5. The test element (110) according to claim 4, wherein said thrombin-like serine protease is a batroxobin.

6. The test element (110) according to claim 1, wherein the detector compound (126) is disposed on the test electrodes (122).

7. The test element (110) according to claim 1, wherein the detector compound (126) is comprised in at least one detector compound layer (128).

8. The test element (110) according to claim 1, wherein the test element (110) further comprises at least one cover layer (144) and at least one spacer layer (142) disposed between the substrate (114) and the cover layer (144), wherein the spacer layer (142) forms sidewalls (164) of the capillary (116).

9. The test element (110) according to claim 1, wherein the test electrodes (122) contain test electrode fingers (132) spaced apart in a direction of longitudinal extension of the capillary (116), and wherein the control electrodes (124) contain control electrode fingers (134) spaced apart in a direction of longitudinal extension of the capillary (116).

10. The test element (110) according to claim 1, wherein the test electrodes (122) comprise two test electrodes (122), the two test electrodes (122) having interleaving test electrode fingers (132), and wherein the control electrodes (124) comprise two control electrodes (124), the two control electrodes (124) having interleaving control electrode fingers (134).

11. An impedance test system (146) for detecting a fibrinogen level in a sample (112) of a bodily fluid, the impedance test system (146) comprising:
a) at least one test element (110) according to claim 1; and
b) at least one test device (148) having contacts (150) for electrically contacting the test element (110), the test device (148) further having at least one impedance measurement unit (152), wherein the impedance measurement unit (152) is configured for performing at least one electrical impedance test measurement using the test electrodes (122), wherein the impedance measurement unit (152) is further configured for performing at least one electrical impedance control measurement using the control electrodes (124).

12. The impedance test system (146) according to claim 11, the impedance test system (146) further having at least one evaluation unit, the evaluation unit being configured for determining the fibrinogen level by using measurement results from the electrical impedance test measurement and from the electrical impedance control measurement.

13. An impedance method of ex-vivo detecting a fibrinogen level in a sample (112) of a bodily fluid, comprising
i) providing at least one test element (110) according to claim 1;
ii) applying the sample (112) of the bodily fluid to at least one application opening (130) of the capillary (116);
iii) performing at least one electrical impedance test measurement by using the test electrodes (122);
iv) performing at least one electrical impedance control measurement using the control electrodes (124); and
v) determining the fibrinogen level by using measurement results from the electrical impedance test measurement and from the electrical impedance control measurement.

14. A resistance test system (216) for detecting a fibrinogen level in a sample of a bodily fluid (112), the test system (216) comprising:
a) at least one test element (110) according to claim 1; and
b) at least one test device (218) having contacts (220) for electrically contacting the test element (110), the test device (218) further having at least one voltage measurement unit (222), wherein the voltage measurement unit (222) is configured for applying at least one voltage signal to the test electrodes (122) and the control electrodes (124) of the test element (110), wherein the voltage measurement unit (222) is further configured for determining at least one test response signal of the test region (118), wherein the voltage measurement unit (222) is further configured for determining at least one control response signal of the control region (120).

15. A resistance method of ex-vivo detecting a fibrinogen level in a sample of a bodily fluid (112), comprising:
i) providing at least one test element (110) according to claim 1;
ii) applying the sample of the bodily fluid (112) to at least one application opening (130) of the capillary (116);
iii) applying at least one voltage signal to the test electrodes (122) and the control electrodes (124) of the test element (110);
iv) determining at least one test response signal of the test region (118) and at least one control response signal of the control region (120); and
v) determining the fibrinogen level by using the test response signal and the control response signal.

16. A method of manufacturing a test element (110) for detecting a fibrinogen level in a sample (112) of a bodily fluid, the method comprising:
i) providing at least one substrate (114) having disposed thereon at least two test electrodes (122) and at least two control electrodes (124);
ii) disposing at least one detector compound (126) being capable of specifically cleaving fibrinogen;
iii) providing at least one spacer layer (142);
iv) providing at least one cover layer (144); and
v) forming a sandwich setup (138) comprising, in the given order, the substrate (114), the spacer layer (142) and the cover layer (144), wherein a capillary (116) is provided in the sandwich setup (138) having sidewalls (164) formed by the spacer layer (142) with the test electrodes (122) and the control electrodes (124) facing the capillary (116), wherein at least one test region (118) and at least one control region (120) are formed within the capillary (116), wherein the test electrodes (122) are disposed within the test region (118), wherein the control electrodes are disposed within the control region (120), and wherein the detector compound (126) is disposed within the test region (118).
